(19) United States
(12) Patent Application Publication
(10) Pub. No.: US 2024/0091266 A1
(43) Pub. Date: Mar. 21, 2024

(54) OPIOID SPARING COMPOSITIONS AND METHODS OF USING THE SAME

(71) Applicants: Silviu ITESCU, Melbourne, Victoria (AU); Roger BROWN, New York, NY (US)

(72) Inventors: Silviu ITESCU, Melbourne, Victoria (AU); Roger BROWN, New York, NY (US)

(21) Appl. No.: 18/262,505

(22) PCT Filed: Jan. 21, 2022
(86) PCT No.: PCT/US2022/013349

§ 371 Date: Jul. 21, 2023

Related U.S. Application Data
(60) Provisional application No. 63/298,391, filed on Jan. 11, 2022.

(30) Foreign Application Priority Data
Jan. 22, 2021 (AU) 2021900147
Feb. 4, 2021 (AU) 2021900250

(51) Int. Cl. A61K 35/28; A61K 47/36; A61P 25/04

(52) U.S. Cl. CPC A61K 35/28; A61K 47/36; A61P 25/04

(57) ABSTRACT

The present disclosure relates to opioid sparing compositions and use of the same for reducing opioid use. Such compositions and methods method may be used for reducing pain or reducing opioid use or increasing mobility in subjects using an opioid.

### CROSS REFERENCE TO RELATED APPLICATIONS

This application claims the priority benefit of Australian Application No. 2021900147, filed Jan. 22, 2021, Australian Application No. 2021900250, filed Feb. 4, 2021, and U.S. Application No. 63/298,391, filed Jan. 11, 2022, each of which is herein incorporated by reference in its entirety.

### TECHNICAL FIELD

The present disclosure relates to opioid sparing compositions and use of the same for reducing opioid use. Such compositions and methods method may be used for reducing pain or reducing opioid use or increasing mobility in subjects using an opioid.

### BACKGROUND

Opioids, also known as opioid agonists, are a group of drugs that exhibit opium or morphine-like properties. The opioids are employed primarily as moderate to strong analgesics, but have many other pharmacological effects as well, including drowsiness, respiratory depression, changes in mood and mental clouding without a resulting loss of consciousness. Prolonged opioid use is associated with various types of pain.

Lower back pain is a chronic condition associated with inflammation and pain requiring ongoing opioid use as part of routine pain management. This condition affects approximately two-thirds of the U.S. adult population, leads to significant increases in physician office visits, and has a significant effect on disability.

Although a variety of therapeutic agents have been used for treating pain and/or inflammation, including chronic pain and/or inflammation, the treatment is often still ineffective even following chronic administration of such agents.

Clearly, there is a need in the art for improved methods of managing pain in opioid users.

### SUMMARY

The present inventors have surprisingly found that administering mesenchymal lineage precursor or stem cells (MLPSC)s can reduce pain or, reduce opioid use or, increase EQ-5D scores in subjects using an opioid. Accordingly, in an example, the present disclosure relates to a method of reducing pain or reducing opioid use or increasing EQ-5D score, the method comprising administering to the subject a composition comprising mesenchymal lineage precursor or stem cells (MLPSCs), wherein the subject is using an opioid. For example, the present disclosure relates to a method of reducing pain, the method comprising administering to the subject a composition comprising mesenchymal lineage precursor or stem cells (MLPSCs), wherein the subject is using an opioid. In another example, the present disclosure relates to method of reducing opioid use, the method comprising administering to the subject a composition comprising mesenchymal lineage precursor or stem cells (MLPSCs), wherein the subject is using an opioid. In another example, the present disclosure relates to method of increasing EQ-5D score, the method comprising administering to the subject a composition comprising mesenchymal lineage precursor or stem cells (MLPSCs), wherein the subject is using an opioid.

In an example, the composition comprises hyaluronic acid (HA). In an example, the opioid sparred by the composition is morphine.

In an example, the methods of the present disclosure reduce pain and opioid use. In another example, the methods of the present disclosure reduce pain and improve function while also reducing opioid use.

In an example, the subject is using an opioid for pain. In an example, the pain is axial pain. In an example, the axial pain is due to nerve root compression defined by MRI.

In view of the present inventors findings, they have arrived at an opioid sparing composition that can be administered to reduce opioid use over time. Accordingly, in another example, the present disclosure relates to an opioid sparing composition comprising mesenchymal lineage precursor or stem cells (MLPSCs). In an example, the composition also comprises hyaluronic acid (HA). For example, the composition may comprise 1% HA. In an example, the composition comprises 1% HA and culture expanded MLPSCs. In an example, the opioid sparing composition is administered to a subject using an opioid, preferably, wherein the subject is using the opioid for pain. In an example, the opioid sparring composition reduces opioid use by a subject. In an example, opioid use is reduced 36 months after administration of the composition. In an example, the opioid sparring composition discontinues opioid use by a subject. In an example, opioid use is discontinued 36 months after administration of the composition.

Compositions and methods of the disclosure can be used in the context of varying types of pain. In an example, the pain is chronic pain. In another example, the pain is lower back pain. In an example, the lower back pain is associated with a degenerated disc. In another example, the lower back pain is associated with an intervertebral disc. For example, the disc can have a disc height that is not substantially reduced compared to that of an adjacent healthy disc in the subject. In another example, the lower back pain is non-radicular in origin. In another example, the pain is associated with one or more of an intervertebral disc herniation up to a 3 mm protrusion; nerve ingrowth into an intervertebral disc; or, inflammation in an intervertebral disc. In an example, the nerve ingrowth or inflammation is in the intervertebral disc space, or the nucleus pulposus, or the annulus fibrosis of the intervertebral disc.

In an example, opioid use is reduced 1 month after administration of the composition. In another example, opioid use is reduced 3 months after administration of the composition. In another example, opioid use is reduced 12 months after administration of the composition. In another example, opioid use is reduced 24 months after administration of the composition. In another example, opioid use is reduced 36 months after administration of the composition. In another example, opioid use is discontinued 36 months after administration of the composition.

In an example, opioid use is reduced relative to the subjects average baseline morphine equivalent dose prior to administering the composition.

In an example, the subject has been using an opioid for at least 1 month prior to administering the composition. In another example, the subject has been using an opioid for at least 3 month prior to administering the composition. In another example, the subject has been using an opioid for at least 6 month prior to administering the composition.

In an example, the subject has been experiencing pain for between 6 and 68 months. In another example, the subject has not been experiencing pain for more than 68 months.

In an example, the subjects opioid use is reduced by about 20% relative to their baseline opioid use prior to administering the composition. In another example, the subjects opioid use is reduced by about 30% relative to their baseline opioid use prior to administering the composition. In another example, the subjects opioid use is reduced by about 40% relative to their baseline opioid use prior to administering the composition.

In an example, the subjects mean visual analogue scale (VAS) score is reduced relative to their VAS score prior to administering the composition. In another example, the subjects VAS pain response is 30%. In another example, the subjects VAS pain response is 50%.

In an example, the subjects mean EQ-5D score is increased from baseline. In an example, the increase is determined based on EQ-5D VAS score. In an example, the increase is observed 12 months after administration of a composition of the disclosure. In an example, the increase is observed 36 months after administration of a composition of the disclosure. In an example, the increase is determined based on EQ-5D index score. In an example, the increase is observed 12 months after administration of a composition of the disclosure. In another example, the increase is observed 24 months after administration of a composition of the disclosure. In another example, the increase is observed 36 months after administration of a composition of the disclosure.

In an example, the subject achieves a ODI 10 point function response. In another example, the subject achieves a ODI 15 point function response. In another example, the subjects achieves a ODI 10 point function response and a VAS pain response of 30%. In another example, the subjects achieves a ODI 15 point function response and a VAS pain response of 50%. In an example, these subjects are administered a composition comprising MLPSCs and HA. In an example, the subject achieves a ODI 10 point function response 12 months after administration of a composition of the disclosure. In an example, the subject is not using opioids prior to administering the composition. In an example, the subject achieves a ODI 10 point function response 36 months after administration of a composition of the disclosure. In an example, the subject is using opioids prior to administering the composition.

In another example, the subject achieves a ODI 10 point function response and a VAS pain response of 30%, wherein the subject is using opioids prior to administering a composition of the disclosure and the subject has not reported lower back pain for more than 68 months. In an example, the ODI 10 point function response and a VAS pain response of 30% is observed 24 months after administration of a composition of the disclosure. In an example, the ODI 10 point function response and a VAS pain response of 30% is observed 36 months after administration of a composition of the disclosure.

In an example, the subject has no significant pain 12 months after administering the composition. In another example, the subject has no significant pain 24 months after administering the composition.

In an example, the subject has an ODI score between 25 and 70%. In another example, the subject has an ODI score between 30 and 60%.

In an example, the MLPSCs are STRO-1+. In an example, the MLPSCs are mesenchymal stem cells (MSCs). In an example, the cells are allogeneic. In an example, the cells are culture expanded. In an example, the cells are TNAP+ before they are culture expanded. In an example, the cells have been cryopreserved.

In an example, methods of the disclosure comprise administering between 1×10⁷ and 2×10⁸ cells. Accordingly, compositions of the disclosure may comprise between 1×10⁷ and 2×10⁸ cells.

In an example, compositions of the disclosure comprise human bone marrow-derived allogeneic mesenchymal precursor cells (MPCs) isolated from bone mononuclear cells with anti-STRO-3 antibodies, expanded ex vivo, and cryopreserved.

In an example, the subject discontinues opioid use 36 months after administering the composition.

In an another example, the present disclosure relates to the manufacture of a medicament for discontinuing opioid use in a subject. In an example, a composition disclosed herein in used in the manufacture of the medicament. In an example, opioid use is discontinued 36 months after administration of the medicament to a subject disclosed herein. For example, the medicament may be administered to a subject that has chronic pain. In an example, the subject has lower back pain.

### BRIEF DESCRIPTION OF DRAWINGS

FIG. 1: LS Mean VAS Pain Change from Baseline in Opioid Users MPC+HA Had Significantly Greater Mean Pain Reduction From Baseline At Every Time Point Over 24 Months Compared With Placebo.

FIG. 2: 50% and 30% VAS Pain Response in Opioid Users MPC+HA Significantly Increased Proportion of Patients At 24 Months With 50% and 30% VAS Pain Response Compared With Placebo.

FIG. 3: Minimal to No Pain (LBP VAS ≤20) in Opioid Users MPC+HA Significantly Increased Proportion of Patients With Minimal/No Pain at 6, 12, 18 and 24 Months Compared With Placebo.

FIG. 4: Comparison of ODI and EQ5D Index As Measures of Function in Opioid Users Mean Change From Baseline In EQ5D Index Better Discriminator of MPC Treatment Effect.

FIG. 5: EQ5D Better Discriminator Than ODI for Determining MIC Treatment Success in Opioid Users.

FIG. 6: MPC+HA Significantly Increased Proportion of Patients at 24 Months Achieving Composite Treatment Responses 50% VAS/0.03EQ5D and 30% VAS/0.03EQ5D.

FIG. 7: LBP-EQ5D Index Overall Treatment Success & MIC Overall Treatment Success in Opioid Users MPC+HA Significantly Increased Proportion of Patients Achieving Composite Treatment Responses 30% VAS/0.03EQ5D at BOTH 12 and 24 Months.

FIG. 8. LBP-EQ5D Index Overall Treatment Success & MIC Overall Treatment Success in Opioid Users MPC+HA Significantly Increased Proportion of Patients Achieving Composite Treatment Responses 30% VAS/0.03EQ5D at BOTH 12 and 24 Months.

FIG. 9. MPC+HA Significantly Reduces Morphine Milligram Equivalent Use of Opioids Over 24 Months In Patients Using Opioids at Baseline.

FIG. 10. MPC+HA Reduces Morphine Milligram Equivalents Over 24 Months in Opioid Users.

FIG. 11. MPC+HA: VAS 30%+opioid reduction*. (*All subjects are opioid users at baseline and have an opioid value at each time point assessed).

FIG. 12. MPC+HA: VAS 50%+opioid reduction*.

FIG. 13. MPC+HA: Composite response; ODI-10+opioid reduction*.

FIG. 14. MPC+HA: Composite response; ODI-15+opioid reduction*.

FIG. 15. MPC+HA: Composite response; VAS 30%+ODI-10+opioid reduction*.

FIG. 16. MPC+HA: Composite response; VAS 50%+ODI-15+opioid reduction*.

FIG. 17. MPC+HA: Treatment success; VAS+ODI response at both 12 and 24 months+opioid reduction* from baseline and no intervention. Treatment Success VAS/ODI: Subjects with a 50% reduction in VAS score from baseline AND 15-point improvement in ODI score from baseline at both 12 & 24 months AND no post-treatment intervention through 24 months AND opioid reduction at 24 months. Subjects with a 30% reduction in VAS score from baseline AND 10-point improvement in ODI score from baseline at both 12 & 24 months AND no post-treatment intervention through 24 months AND opioid reduction at 24 months.

FIG. 18. MPC+HA: Treatment success; VAS+EQ5D response at both 12 and 24 months+opioid reduction* from baseline and no intervention. Treatment Success VAS/EQ5D Index: subjects with a 50% reduction in VAS score from baseline AND 0.03 point improvement in EQ5D Index score from baseline at both 12 & 24 months AND no post-treatment intervention through 24 months AND opioid reduction at 24 months. Subjects with a 30% reduction in VAS score from baseline AND 0.03 point improvement in EQ5D Index score from baseline at both 12 & 24 months AND no post-treatment intervention through 24 months AND opioid reduction at 24 months.

FIG. 19. LS Mean VAS Low Back Pain Change From Baseline—All Subjects (n=391).

FIG. 20. LS Mean VAS Low Back Pain Change From Baseline—CLBP<Median (n=194).

FIG. 21. LS Mean ODI Change From Baseline—CLBP<Median (n=197).

FIG. 22. LHS: ODI 10-point MIC responders—CLBP≤Median; RHS: MIC 30% VAS/10-point ODI treatment success—CLBP≤Median.

FIG. 23. EQ5D Index CFB—All subjects (LHS); CLBP duration<Median (RHS).

FIG. 24. EQ5D MCIC Index responders—All subjects (LHS); CLBP duration<Median (RHS).

FIG. 25. 30% VAS-EQ5D 0.03 point Index MIC treatment success—All subjects (LHS); CLBP duration<Median (RHS).

FIG. 26. 50% VAS-EQ5D 0.03 point Index MIC treatment success—All subjects (LHS); CLBP duration<Median (RHS).

FIG. 27. LHS: VAS 30%/ODI 10% MIC treatment success CLBP duration<median; RHS: VAS 30%/EQ5d 0.03 point MIC treatment success CLBP<median. ODI MIC Treatment Success—Subjects with a 30% reduction in Low Back Pain VAS score from baseline AND 10-point improvement in ODI from baseline AND no post-treatment intervention through time point being evaluated. EQ5D Index Score MIC Treatment Success—Subjects with a 30% reduction in Low Back Pain VAS score from baseline AND 0.03-point improvement in EQ5D Index score from baseline AND no post-treatment intervention through time point being evaluated.

FIG. 28. LS mean change in average pain over 24 hours measured by VAS—Pre-specified subgroup of baseline opioid users based upon patient E-diary pain medication use entries.

FIG. 29. LS mean change in average pain over 24 hours measured by VAS—all subjects.

FIG. 30. Proportion of subjects at 24 Months with an average pain medication reduction from baseline.

FIG. 31. Proportion of baseline opioid users without opioid use at 6, 12, 18, 24 and 36 Months.

FIG. 32. Average pain minimally important change (MIC; 30% Reduction) responder analyses—Pre-specified subgroup of subjects with duration CLBP less than median (68.8 Months).

FIG. 33. LS mean change in function measured by ODI—Subjects with duration CLBP less than median for opioid users and opioid non-users through 36 months.

FIG. 34. LS mean change in morphine equivalent dose for opioid users through 36 months.

### DETAILED DESCRIPTION

### General Techniques and Definitions

Unless specifically defined otherwise, all technical and scientific terms used herein shall be taken to have the same meaning as commonly understood by one of ordinary skill in the art (e.g., in cell culture, molecular biology, stem cell culture, immunology, and biochemistry).

Unless otherwise indicated, cell culture techniques and assays utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning.

As used herein, the term “about”, unless stated to the contrary, refers to +/−10%, more preferably +/−5%, of the designated value.

The terms “level” and “amount” are used to define the amount of a particular substance in a sample from a subject or in a cell culture media (or sample therefrom). For example, a particular concentration, weight, percentage (e.g. v/v %) or ratio can be used to define the level of a particular substance in a sample. In an example, the level is expressed in terms of how much of a particular marker is expressed by cells of the disclosure under culture conditions. In an example, expression represents cell surface expression. In another example, the level is expressed in terms of how much of a particular marker is released from cells described herein under culture conditions. In an example, the sample is obtained from a patient or subject (e.g. a blood sample) and the level of a substance is measured in the sample to determine the level of the substance in the sample.

In an example, the level is expressed in pg/ml. In another example, the level is expressed in pg per 10⁶ cells.

In an example, the level of a particular marker in a cell culture medium is determined under culture conditions. The term “culture conditions” is used to refer to cells growing in culture. In an example, culture conditions refers to an actively dividing population of cells. Such cells may, in an example, in exponential growth phase. For example, the level of a particular marker can be determined by taking a sample of cell culture media and measuring the level of marker in the sample. In another example, the level of a particular marker can be determined by taking a sample of cells and measuring the level of the marker in the cell lysate. Those of skill in the art that secreted markers will be measured by sampling the culture media while markers expressed on the surface of the cell may be measured by assessing a sample of cell lysate. In an example, the sample is taken when the cells are in exponential growth phase. In an example, the sample is taken after at least two days in culture.

Culture expanding cells from a cryopreserved intermediate means thawing cells subject to cryogenic freezing and in vitro culturing under conditions suitable for growth of the cells.

In an example, the “level” or “amount” of a particular marker is determined after cells have been cryopreserved and then seeded back into culture. For example, the level is determined after a first cryopreservation of cells. In another example, the level is determined after a second cryopreservation of cells. For example, cells may be culture expanded from a cryopreserved intermediate, cryopreserved a second time before being re-seeded in culture so that the level of a particular marker can be determined under culture conditions.

The term “opioid sparing agent” is used in the context of the present disclosure to refer to a composition that can be administered to a subject using an opioid in order to facilitate reduction of the subjects opioid intake. In other words, subjects administered an opioid sparing agent of the disclosure are able to take a lower dose of opioid over time. In an example, the opioid sparing agent reduces a subjects pain and/or increases their mobility.

As used herein, the terms “treating”, “treat”, “treatment”, “reducing pain”, “reducing opioid use”, “increasing mobility” include administering a population of mesenchymal lineage stem or precursor cells and/or progeny thereof and/or soluble factors derived therefrom and/or extracellular vesicles derived therefrom to thereby reduce pain and/or reduce opioid use and/or increase mobility in a subject using an opioid.

The term “subject” as used herein refers to a human subject. For example, the subject can be an adult. In another example, the subject can be a child. In another example, the subject can be an adolescent. Terms such as “subject”, “patient” or “individual” are terms that can, in context, be used interchangeably in the present disclosure. Subjects in need of treatment include subjects using an opioid. For example, the subject can be using an opioid for pain such as lower back pain. In an example, the subjects pain and opioid use is ongoing. For example, the subject can have pain for greater than 2, 3, 4, 5, 6, 12, 24 months or more. Accordingly, in an example, the subject can be using an opioid for greater than 1, 2, 3, 4, 5, 6, 12, 24 months or more. In an example, the subject is using morphine. In an example, the subject has not reported lower back pain for more than 68 months.

In another example, the subject has an Oswestry Disability Index (ODI) score between 25 and 70%. In another example, the subject has an ODI score between 30 and 60%. ODI scores can be used to define patients as follows: 0%-20%: Minimal disability; 21%-40%: Moderate Disability; 41%-60%: Severe Disability; 61%-80%: Crippling back pain; 81%-100%: either bed-bound or have an exaggeration of their symptoms. In an example, the subject has been experiencing pain for at least a month. In another example, the subject has been experiencing pain for at least 3 months. In another example, the subject has been experiencing pain for at least 6 months. In another example, the subject has been experiencing pain for at least 12 months. In another example, the subject has been experiencing pain for at least 24 months. In another example, the subject has been experiencing pain for at least 36 months. In another example, the subject has been experiencing pain for at least 48 months. In another example, the subject has been experiencing pain for at least 60 months. In another example, the subject has been experiencing pain for between 6 and 78 months. In another example, the subject has been experiencing pain for between 6 and 70 months. In another example, the subject has been experiencing pain for between 2 and 68 months. In another example, the subject has been experiencing pain for between 3 and 68 months. In another example, the subject has been experiencing pain for between 6 and 68 months. In another example, the subject has been experiencing pain for between 12 and 68 months. In another example, the subject has not been experiencing pain for more than 68 months. In an example, the subjects pain requires treating with an analgesic. In an example, the analgesic is an opioid. In an example, the opioid is morphine. For example, the subject may have been experiencing chronic lower back pain for between 3 and 68 months. In this example, the subjects pain may have been treated with an opioid. In an example, the subject has been experiencing chronic lower back pain for at least 6 months. In an example, the subject has been experiencing chronic lower back pain for 6 to 12 months.

In certain embodiments, the present disclosure relates to methods and compositions for reducing pain such as chronic lower back pain. Reductions in pain can be determined using various methods as discussed herein (e.g. VAS; EQ-5D; ODI). The term “axial pain” is used in the context of the present disclosure to refer to localised pain, confined to a certain spot or region (e.g. neck pain or leg pain).

In an example, compositions of the disclosure comprise genetically unmodified mesenchymal precursor lineage or stem cells. As used herein, the term “genetically unmodified” refers to cells that have not been modified by transfection with a nucleic acid. For the avoidance of doubt, in the context of the present disclosure a mesenchymal lineage precursor or stem cell transfected with a nucleic acid encoding a protein would be considered genetically modified.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Those skilled in the art will appreciate that the disclosure described herein is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific embodiments described herein, which are intended for the purpose of exemplification only. Functionally-equivalent products, compositions and methods are clearly within the scope of the disclosure, as described herein.

Any example disclosed herein shall be taken to apply mutatis mutandis to any other example unless specifically stated otherwise.

### Opioid Use

The methods of the present disclosure are directed towards subjects using an opioid. In an example, the subjects are using an opioid for pain. In an example, the pain is chronic pain. For example, the pain can be lower back pain. Opioids are a group of pain-relieving drugs that exhibit opium or morphine-like properties. In an example, opioids may be referred to as opioid receptor agonists.

Exemplary opioids include alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphine, normorphine, norpipanone, opium, oxycodone, oxymorphone, papaveretum, pentazocine, phenadoxone, phenomorphan, phenazocine, phenoperidine, piminodine, piritramide, propheptazine, promedol, properidine, propoxyphene, sufentanil, tilidine, tramadol, mixtures of any of the foregoing, salts of any of the foregoing, and the like. In an example, the opioid is morphine or an analogue thereof that interacts with the morphine receptor.

In an example, the subject has been using an opioid for at least a month. In another example, the subject has been using an opioid for at least 3 months. In another example, the subject has been using an opioid for at least 6 months. In another example, the subject has been using an opioid for at least 12 months. In another example, the subject has been using an opioid for at least 24 months. In another example, the subject has been using an opioid for at least 36 months. In another example, the subject has been using an opioid for at least 48 months. In another example, the subject has been using an opioid for at least 60 months. In another example, the subject has been using an opioid for between 6 and 78 months. In another example, the subject has been using an opioid for between 6 and 70 months. In another example, the subject has been using an opioid for between 6 and 68 months. In another example, the subject has not been using an opioid for more than 68 months.

In an example, the subject has an average baseline morphine equivalent dose (MED) of <75 mg/day. In an example, the MED is determined by e-diary entries from the subject.

In another example, the subject is using an opioid for chronic low back pain for at least 6 months. In another example, the subject has failed at least 2, 3, 4, 5 or 6 months of conservative back pain care. Examples of conservative treatment regimens may include any or all of the following: initial rest, medications such as anti-inflammatory, analgesics, narcotics/opioids, muscle relaxants or other interventions such as massage, acupuncture, chiropractic manipulations, activity modification, home-directed lumbar exercise program.

In another examples the subject using an opioid has undergone supervised physical therapy, such as daily walking routines, therapeutic exercises, and back education programs specifically for the treatment of low back pain.

In another example, the subject using an opioid has a modified Pfirrmann score of 3, 4, 5 or 6 on MRI at the index disc and/or Modic Grade II changes or less on MRI at the index disc. In an example, the subject using an opioid can be with or without contained disc protrusion at the index disc on MRI.

In an example, the subject using an opioid can have low back pain of at least 40 mm and not more than 90 mm of 100 mm on low back pain VAS (average pain over 24 hours).

In another example, the subject using an opioid has lower back pain is associated with a degenerated disc. In another example, the lower back pain is associated with an intervertebral disc. In an example, the disc has a disc height that is not substantially reduced compared to that of an adjacent healthy disc in the subject. In another example, the lower back pain is non-radicular in origin and/or is associated with one or more of an intervertebral disc herniation up to a 3 mm protrusion; nerve ingrowth into an intervertebral disc; inflammation in an intervertebral disc. In an example, the nerve ingrowth or inflammation is in the intervertebral disc space, or the nucleus pulposus, or the annulus fibrosis of the intervertebral disc.

In another example, the pain is axial pain. Accordingly, in an example, the present disclosure relates to a method of treating axial pain in a subject, the method comprising administering a composition of the disclosure. In an example, the subject is using opioids. In an example, the composition comprises MLPSCs and HA. In an example, the axial pain is leg pain. In an example, the axial pain is due to nerve root compression defined by MRI. In an example, the subject has not reported chronic pain for more than 68 months. In another example, the subject has not been using opioids for more than 68 months.

### Reducing Pain or Reducing Opioid Use or Increasing Mobility

In an example, the methods of the present disclosure reduce pain in a subject using an opioid. In an example, administering a composition of the disclosure reduces the subjects visual analogue scale (VAS) pain response. A VAS is a measurement instrument that is commonly used to measure pain because it assists in measuring a characteristic or attitude that is believed to range across a continuum of values that cannot easily be directly measured. In short, the pain VAS is a unidimensional measure of pain intensity. The patient marks on the line the point that they feel represents their perception of their current pain state. The VAS score is determined by measuring in millimetres from the left hand end of the line to the point that the patient marks. VAS scores can be compared over time to measure changes in pain levels. Accordingly, it can be useful indicator of a reduction in pain. In an example, administering a composition of the disclosure reduces the subjects visual analogue scale (VAS) pain response by 10%. In another example, administering a composition of the disclosure reduces the subjects VAS by 20%. In another example, administering a composition of the disclosure reduces the subjects VAS by 30%. In another example, administering a composition of the disclosure reduces the subjects VAS by 50%. In these examples, the reduction in VAS may be determined via comparison with a baseline VAS score obtained prior to a treatment disclosed herein. In an example, the reduction in VAS is observed 12 months after treatment. In another example, the reduction in VAS is observed 24 months after treatment.

In an example, the subject has no significant pain after administering a composition of the disclosure. In an example, the subject has no significant pain 12 months after administering a composition of the disclosure. In another example, the subject has no significant pain 24 months after administering a composition of the disclosure.

The present inventors have identified that compositions of the disclosure can be used as opioid sparing agents. Accordingly, in another example, the methods of the present disclosure reduce opioid use in a subject. In an example, opioid use is reduced 1 month after administration of the composition. In another example, opioid use is reduced 3 month after administration of the composition. In another example, opioid use is reduced 6 month after administration of the composition. In another example, opioid use is reduced 12 month after administration of the composition. In another example, opioid use is reduced 18 month after administration of the composition. In another example, opioid use is reduced 24 month after administration of the composition. In another example, opioid use is reduced 36 month after administration of the composition. In an example, the subjects opioid use is reduced relative to their baseline opioid use prior to administering a composition of the disclosure. For example, the subjects opioid use can be reduced relative to the subjects average baseline morphine equivalent dose prior to administering the composition.

In an example, the subjects opioid use is reduced by about 20% relative to their baseline opioid use prior to administering the composition. In another example, the subjects opioid use is reduced by about 30% relative to their baseline opioid use prior to administering the composition. In another example, the subjects opioid use is reduced by about 40% relative to their baseline opioid use prior to administering the composition. In an example, the opioid is morphine and the reduction is relative to the subjects average baseline morphine equivalent dose prior to administering the composition.

In an example, the subject discontinues opioid use after administering a composition of the disclosure. In an example, the subject discontinues opioid use 12 months after administering a composition of the disclosure. In another example, the subject discontinues opioid use 18 months after administering a composition of the disclosure. In another example, the subject discontinues opioid use 24 months after administering a composition of the disclosure. In another example, the subject discontinues opioid use 36 months after administering a composition of the disclosure. In another example, the subject discontinues opioid use between 18 and 36 months after administering a composition of the disclosure.

In an example, the methods of the present disclosure increase mobility and/or function in a subject using an opioid. In an example, mobility and/or function is described as a subjects function according to well established measures of this criteria such as ODI and/or EQ-5D. In an example, administering a composition of the disclosure increases the subjects EuroQol-5 Dimension (EQ-5D) score from baseline. Accordingly, in an example, the methods of the present disclosure increase EQ-5D score in a subject using an opioid. The EQ-5D provides a simple descriptive profile and a single index value for health status. The EQ-5D self-reported questionnaire includes a visual analogue scale (VAS), which records the respondent's self-rated health status on a graduated (0-100) scale, with higher scores for higher health-related quality of life. In certain examples, EQ-5D also includes the EQ-5D descriptive system, which comprises 5 dimensions of health: mobility, self-care, usual activities, pain/discomfort, and anxiety/depression. This descriptive system can be used as a health profile or converted into an index score representing a von Neumann-Morgenstern utility value for current health (EQ-5D index).

In an example, the EQ-5D is increased for at least 1 to 6 months. In an example, the EQ-5D is increased for at least 6 to 12 months. In an example, EQ-5D is increased by at least 0.01. In an example, EQ-5D is increased by at least 0.02. In an example, EQ-5D is increased by at least 0.03. In an example, EQ-5D is increased between 0.01 and 0.04. In an example, the increase is determined based on EQ-5D VAS.

In another example, administering a composition of the disclosure can reduce a subjects ODI score. In an example, the ODI is reduced by at least 10 or at least 15 points for at least 1 to 6 months. In an example, the ODI is reduced by at least 10 or at least 15 points for at least 6 to 12 months. In another example, the ODI is reduced by at least 10 or at least 15 points for at least 24 months.

In another example, the methods of the present disclosure relate to treating a subject, the method comprising administering a composition according to the disclosure and an opioid, wherein multiple doses of the opioid are administered to the subject and the dose of opioid is decreased across the course of treatment. For example, the dose of opioid can be decreased by around 20-50% over the course of treatment following administration of a composition of the disclosure.

In another example, the present disclosure relates to a method of reducing opioid use in a subject, the method comprising administering a composition of the disclosure. For example, the method can comprise administering a composition comprising MLPSCs and HA. In an example, the subject has chronic lower back pain. In an example, the subject has not had chronic lower back pain for more than 68 months. In an example, opioid use is reduced between 12 to 36 months after administering a composition of the disclosure. In an example, the reduction in opioid use is sustained 36 months after administering a composition of the disclosure. In an example, opioid use is discontinued 18 to 36 months after administering a composition of the disclosure.

### Mesenchymal Lineage Precursor Cells

As used herein, the term “mesenchymal lineage precursor or stem cell (MLPSC)” refers to undifferentiated multipotent cells that have the capacity to self-renew while maintaining multipotency and the capacity to differentiate into a number of cell types either of mesenchymal origin, for example, osteoblasts, chondrocytes, adipocytes, stromal cells, fibroblasts and tendons, or non-mesodermal origin, for example, hepatocytes, neural cells and epithelial cells. For the avoidance of doubt, a “mesenchymal lineage precursor cell” refers to a cell which can differentiate into a mesenchymal cell such as bone, cartilage, muscle and fat cells, and fibrous connective tissue.

The term “mesenchymal lineage precursor or stem cells” includes both parent cells and their undifferentiated progeny. The term also includes mesenchymal precursor cells, multipotent stromal cells, mesenchymal stem cells (MSCs), perivascular mesenchymal precursor cells, and their undifferentiated progeny.

Mesenchymal lineage precursor or stem cells can be autologous, allogeneic, xenogenic, syngeneic or isogenic. Autologous cells are isolated from the same individual to which they will be reimplanted. Allogeneic cells are isolated from a donor of the same species. Xenogenic cells are isolated from a donor of another species. Syngenic or isogenic cells are isolated from genetically identical organisms, such as twins, clones, or highly inbred research animal models.

In an example, the mesenchymal lineage precursor or stem cells are allogeneic. In an example, the allogeneic mesenchymal lineage precursor or stem cells are culture expanded and cryopreserved.

Mesenchymal lineage precursor or stem cells reside primarily in the bone marrow, but have also shown to be present in diverse host tissues including, for example, cord blood and umbilical cord, adult peripheral blood, adipose tissue, trabecular bone and dental pulp. They are also found in skin, spleen, pancreas, brain, kidney, liver, heart, retina, brain, hair follicles, intestine, lung, lymph node, thymus, ligament, tendon, skeletal muscle, dermis, and periosteum; and are capable of differentiating into germ lines such as mesoderm and/or endoderm and/or ectoderm. Thus, mesenchymal lineage precursor or stem cells are capable of differentiating into a large number of cell types including, but not limited to, adipose, osseous, cartilaginous, elastic, muscular, and fibrous connective tissues. The specific lineage-commitment and differentiation pathway which these cells enter depends upon various influences from mechanical influences and/or endogenous bioactive factors, such as growth factors, cytokines, and/or local microenvironmental conditions established by host tissues.

The terms “enriched”, “enrichment” or variations thereof are used herein to describe a population of cells in which the proportion of one particular cell type or the proportion of a number of particular cell types is increased when compared with an untreated population of the cells (e.g., cells in their native environment). In one example, a population enriched for mesenchymal lineage precursor or stem cells comprises at least about 0.1% or 0.5% or 1% or 2% or 5% or 10% or 15% or 20% or 25% or 30% or 50% or 75% mesenchymal lineage precursor or stem cells. In this regard, the term “population of cells enriched for mesenchymal lineage precursor or stem cells” will be taken to provide explicit support for the term “population of cells comprising X % mesenchymal lineage precursor or stem cells”, wherein X % is a percentage as recited herein. The mesenchymal lineage precursor or stem cells can, in some examples, form clonogenic colonies, e.g. CFU-F (fibroblasts) or a subset thereof (e.g., 50% or 60% or 70% or 70% or 90% or 95%) can have this activity.

In an example of the present disclosure, the mesenchymal lineage precursor or stem cells are mesenchymal stem cells (MSCs). The MSCs may be a homogeneous composition or may be a mixed cell population enriched in MSCs. Homogeneous MSC compositions may be obtained by culturing adherent marrow or periosteal cells, and the MSCs may be identified by specific cell surface markers which are identified with unique monoclonal antibodies. A method for obtaining a cell population enriched in MSCs is described, for example, in U.S. Pat. No. 5,486,359. Alternative sources for MSCs include, but are not limited to, blood, skin, cord blood, muscle, fat, bone, and perichondrium. In an example, the MSCs are allogeneic. In an example, the MSCs are cryopreserved. In an example, the MSCs are culture expanded and cryopreserved.

In another example, the mesenchymal lineage precursor or stem cells are CD29+, CD54+, CD73+, CD90+, CD102+, CD105+, CD106+, CD166+, MHC1+ MSCs.

Isolated or enriched mesenchymal lineage precursor or stem cells can be expanded in vitro by culture. Isolated or enriched mesenchymal lineage precursor or stem cells can be cryopreserved, thawed and subsequently expanded in vitro by culture.

In one example, isolated or enriched mesenchymal lineage precursor or stem cells are seeded at 50,000 viable cells/cm² in culture medium (serum free or serum-supplemented), for example, alpha minimum essential media (αMEM) supplemented with 5% fetal bovine serum (FBS) and glutamine, and allowed to adhere to the culture vessel overnight at 37° C., 20% O₂. The culture medium is subsequently replaced and/or altered as required and the cells cultured for a further 68 to 72 hours at 37° C., 5% O₂.

As will be appreciated by those of skill in the art, cultured mesenchymal lineage precursor or stem cells are phenotypically different to cells in vivo. For example, in one embodiment they express one or more of the following markers, CD44, NG2, DC146 and CD140b. Cultured mesenchymal lineage precursor or stem cells are also biologically different to cells in vivo, having a higher rate of proliferation compared to the largely non-cycling (quiescent) cells in vivo.

In one example, the population of cells is enriched from a cell preparation comprising STRO-1+ cells in a selectable form. In this regard, the term “selectable form” will be understood to mean that the cells express a marker (e.g., a cell surface marker) permitting selection of the STRO-1+ cells. The marker can be STRO-1, but need not be. For example, as described and/or exemplified herein, cells (e.g., mesenchymal precursor cells) expressing STRO-2 and/or STRO-3 (TNAP) and/or STRO-4 and/or VCAM-1 and/or CD146 and/or 3G5 also express STRO-1 (and can be STRO-1bright). Accordingly, an indication that cells are STRO-1+ does not mean that the cells are selected solely by STRO-1 expression. In one example, the cells are selected based on at least STRO-3 expression, e.g., they are STRO-3+(TNAP+).

Reference to selection of a cell or population thereof does not necessarily require selection from a specific tissue source. As described herein STRO-1+ cells can be selected from or isolated from or enriched from a large variety of sources. That said, in some examples, these terms provide support for selection from any tissue comprising STRO-1+ cells (e.g., mesenchymal precursor cells) or vascularized tissue or tissue comprising pericytes (e.g., STRO-1+ pericytes) or any one or more of the tissues recited herein.

In one example, the cells used in the present disclosure express one or more markers individually or collectively selected from the group consisting of TNAP+, VCAM-1+, THY-1+, STRO-2+, STRO-4+(HSP-90β), CD45+, CD146+, 3G5+ or any combination thereof.

By “individually” is meant that the disclosure encompasses the recited markers or groups of markers separately, and that, notwithstanding that individual markers or groups of markers may not be separately listed herein the accompanying claims may define such marker or groups of markers separately and divisibly from each other.

By “collectively” is meant that the disclosure encompasses any number or combination of the recited markers or groups of markers, and that, notwithstanding that such numbers or combinations of markers or groups of markers may not be specifically listed herein the accompanying claims may define such combinations or sub-combinations separately and divisibly from any other combination of markers or groups of markers.

As used herein the term “TNAP” is intended to encompass all isoforms of tissue non-specific alkaline phosphatase. For example, the term encompasses the liver isoform (LAP), the bone isoform (BAP) and the kidney isoform (KAP). In one example, the TNAP is BAP. In one example, TNAP as used herein refers to a molecule which can bind the STRO-3 antibody produced by the hybridoma cell line deposited with ATCC on 19 Dec. 2005 under the provisions of the Budapest Treaty under deposit accession number PTA-7282.

Furthermore, in one example, the STRO-1+ cells are capable of giving rise to clonogenic CFU-F.

In one example, a significant proportion of the STRO-1+ cells are capable of differentiation into at least two different germ lines. Non-limiting examples of the lineages to which the STRO-1+ cells may be committed include bone precursor cells; hepatocyte progenitors, which are multipotent for bile duct epithelial cells and hepatocytes; neural restricted cells, which can generate glial cell precursors that progress to oligodendrocytes and astrocytes; neuronal precursors that progress to neurons; precursors for cardiac muscle and cardiomyocytes, glucose-responsive insulin secreting pancreatic beta cell lines. Other lineages include, but are not limited to, odontoblasts, dentin-producing cells and chondrocytes, and precursor cells of the following: retinal pigment epithelial cells, fibroblasts, skin cells such as keratinocytes, dendritic cells, hair follicle cells, renal duct epithelial cells, smooth and skeletal muscle cells, testicular progenitors, vascular endothelial cells, tendon, ligament, cartilage, adipocyte, fibroblast, marrow stroma, cardiac muscle, smooth muscle, skeletal muscle, pericyte, vascular, epithelial, glial, neuronal, astrocyte and oligodendrocyte cells.

In an example, mesenchymal lineage precursor or stem cells are obtained from a single donor, or multiple donors where the donor samples or mesenchymal lineage precursor or stem cells are subsequently pooled and then culture expanded.

Mesenchymal lineage precursor or stem cells encompassed by the present disclosure may also be cryopreserved prior to administration to a subject. In an example, mesenchymal lineage precursor or stem cells are culture expanded and cryopreserved prior to administration to a subject.

In an example, the present disclosure encompasses mesenchymal lineage precursor or stem cells as well as progeny thereof, soluble factors derived therefrom, and/or extracellular vesicles isolated therefrom. In another example, the present disclosure encompasses mesenchymal lineage precursor or stem cells as well as extracellular vesicles isolated therefrom. For example, it is possible to culture expand mesenchymal precursor lineage or stem cells of the disclosure for a period of time and under conditions suitable for secretion of extracellular vesicles into the cell culture medium. Secreted extracellular vesicles can subsequently be obtained from the culture medium for use in therapy.

The term “extracellular vesicles” as used herein, refers to lipid particles naturally released from cells and ranging in size from about 30 nm to as a large as 10 microns, although typically they are less than 200 nm in size. They can contain proteins, nucleic acids, lipids, metabolites, or organelles from the releasing cells (e.g., mesenchymal stem cells; STRO-1⁺ cells).

The term “exosomes” as used herein, refers to a type of extracellular vesicle generally ranging in size from about 30 nm to about 150 nm and originating in the endosomal compartment of mammalian cells from which they are trafficked to the cell membrane and released. They may contain nucleic acids (e.g., RNA; microRNAs), proteins, lipids, and metabolites and function in intercellular communication by being secreted from one cell and taken up by other cells to deliver their cargo.

In an example, compositions of the disclosure comprise cells that induce new blood vessel formation in target tissue.

### Culture Expansion of the Cells

In an example, mesenchymal lineage precursor or stem cells are culture expanded. “Culture expanded” mesenchymal lineage precursor or stem cells media are distinguished from freshly isolated cells in that they have been cultured in cell culture medium and passaged (i.e. sub-cultured). In an example, culture expanded mesenchymal lineage precursor or stem cells are culture expanded for about 4-10 passages. In an example, mesenchymal lineage precursor or stem cells are culture expanded for at least 5, at least 6, at least 7, at least 8, at least 9, at least 10 passages. For example, mesenchymal lineage precursor or stem cells can be culture expanded for at least 5 passages. In an example, mesenchymal lineage precursor or stem cells can be culture expanded for at least 5-10 passages. In an example, mesenchymal lineage precursor or stem cells can be culture expanded for at least 5-8 passages. In an example, mesenchymal lineage precursor or stem cells can be culture expanded for at least 5-7 passages. In an example, mesenchymal lineage precursor or stem cells can be culture expanded for more than 10 passages. In another example, mesenchymal lineage precursor or stem cells can be culture expanded for more than 7 passages. In these examples, stem cells may be culture expanded before being cryopreserved to provide an intermediate cryopreserved MLPSC population. In an example, compositions of the present disclosure are produced by culturing cells from an intermediate cryopreserved MLPSC population or, put another way, a cryopreserved intermediate.

In an example, compositions of the disclosure comprise mesenchymal lineage precursor or stem cells that are culture expanded from a cryopreserved intermediate. In an example, the cells culture expanded from a cryopreserved intermediate are culture expanded for at least 5, at least 6, at least 7, at least 8, at least 9, at least 10 passages. For example, mesenchymal lineage precursor or stem cells can be culture expanded for at least 5 passages. In an example, mesenchymal lineage precursor or stem cells can be culture expanded for at least 5-10 passages. In an example, mesenchymal lineage precursor or stem cells can be culture expanded for at least 5-8 passages. In an example, mesenchymal lineage precursor or stem cells can be culture expanded for at least 5-7 passages. In an example, mesenchymal lineage precursor or stem cells can be culture expanded for more than 10 passages. In another example, mesenchymal lineage precursor or stem cells can be culture expanded for more than 7 passages.

In an example, mesenchymal lineage precursor or stem cells culture expanded from a cryopreserved intermediate can be culture expanded in medium free of animal proteins. In an example, mesenchymal lineage precursor or stem cells culture expanded from a cryopreserved intermediate can be culture expanded in xeno-free medium. In an example, mesenchymal lineage precursor or stem cells culture expanded from a cryopreserved intermediate can be culture expanded in medium that is fetal bovine serum free.

In an embodiment, mesenchymal lineage precursor or stem cells can be obtained from a single donor, or multiple donors where the donor samples or mesenchymal lineage precursor or stem cells are subsequently pooled and then culture expanded. In an example, the culture expansion process comprises:
i. expanding by passage expansion the number of viable cells to provide a preparation of at least about 1 billion of the viable cells, wherein the passage expansion comprises establishing a primary culture of isolated mesenchymal lineage precursor or stem cells and then serially establishing a first non-primary (P1) culture of isolated mesenchymal lineage precursor or stem cells from the previous culture;
ii. expanding by passage expansion the P1 culture of isolated mesenchymal lineage precursor or stem cells to a second non-primary (P2) culture of mesenchymal lineage precursor or stem cells; and,
iii. preparing and cryopreserving an in-process intermediate mesenchymal lineage precursor or stem cells preparation obtained from the P2 culture of mesenchymal lineage precursor or stem cells; and,
iv. thawing the cryopreserved in-process intermediate mesenchymal lineage precursor or stem cells preparation and expanding by passage expansion the in-process intermediate mesenchymal lineage precursor or stem cells preparation.

In an example, the expanded mesenchymal lineage precursor or stem cell preparation has an antigen profile and an activity profile comprising:
i. less than about 0.75% CD45+ cells;
ii. at least about 95% CD105+ cells;
iii. at least about 95% CD166+ cells.

In an example, the expanded mesenchymal lineage precursor or stem cell preparation is capable of inhibiting IL2-Rα expression by CD3/CD28-activated PBMCs by at least about 30% relative to a control.

In an example, culture expanded mesenchymal lineage precursor or stem cells are culture expanded for about 4-10 passages, wherein the mesenchymal lineage precursor or stem cells have been cryopreserved after at least 2 or 3 passages before being further culture expanded. In an example, mesenchymal lineage precursor or stem cells are culture expanded for at least 1, at least 2, at least 3, at least 4, at least 5 passages, cryopreserved and then further culture expanded for at least 1, at least 2, at least 3, at least 4, at least 5 passages before being cultured according to the methods of the disclosure.

The process of mesenchymal lineage precursor or stem cell isolation and ex vivo expansion can be performed using any equipment and cell handing methods known in the art. Various culture expansion embodiments of the present disclosure employ steps that require manipulation of cells, for example, steps of seeding, feeding, dissociating an adherent culture, or washing. Any step of manipulating cells has the potential to insult the cells. Although mesenchymal lineage precursor or stem cells can generally withstand a certain amount of insult during preparation, cells are preferably manipulated by handling procedures and/or equipment that adequately performs the given step(s) while minimizing insult to the cells.

In an example, mesenchymal lineage precursor or stem cells are washed in an apparatus that includes a cell source bag, a wash solution bag, a recirculation wash bag, a spinning membrane filter having inlet and outlet ports, a filtrate bag, a mixing zone, an end product bag for the washed cells, and appropriate tubing, for example, as described in U.S. Pat. No. 6,251,295, which is hereby incorporated by reference.

In an example, a mesenchymal lineage precursor or stem cell composition cultured according to the present disclosure is 95% homogeneous with respect to being CD105 positive and CD166 positive and being CD45 negative. In an example, this homogeneity persists through ex vivo expansion; i.e. though multiple population doublings.

In an example, mesenchymal lineage precursor or stem cells of the disclosure are culture expanded in 3D culture. For example, mesenchymal lineage precursor or stem cells of the disclosure can be culture expanded in a bioreactor. In an example, mesenchymal lineage precursor or stem cells of the disclosure are initially culture expanded in 2D culture prior to being further expanded in 3D culture. In an example, mesenchymal lineage precursor or stem cells of the disclosure are culture expanded from a master cell bank. In an example, mesenchymal lineage precursor or stem cells of the disclosure are culture expanded from a master cell bank in 2D culture before seeding in 3D culture. In an example, mesenchymal lineage precursor or stem cells of the disclosure are culture expanded from a master cell bank in 2D culture for at least 3 days before seeding in 3D culture in a bioreactor. In an example, mesenchymal lineage precursor or stem cells of the disclosure are culture expanded from a master cell bank in 2D culture for at least 4 days before seeding in 3D culture in a bioreactor. In an example, mesenchymal lineage precursor or stem cells of the disclosure are culture expanded from a master cell bank in 2D culture for between 3 and 5 days before seeding in 3D culture in a bioreactor. In these examples, 2D culture can be performed in a cell factory. Various cell factory products are available commercially (e.g. Thermofisher, Sigma).

### Cell Culture Medium

Mesenchymal lineage precursor or stem cells disclosed herein can be culture expanded in various suitable growth mediums.

The term “medium” or “media” as used in the context of the present disclosure, includes the components of the environment surrounding the cells. The media contributes to and/or provides the conditions suitable to allow cells to grow. Media may be solid, liquid, gaseous or a mixture of phases and materials. Media can include liquid growth media as well as liquid media that do not sustain cell growth. Media also include gelatinous media such as agar, agarose, gelatin and collagen matrices. Exemplary gaseous media include the gaseous phase that cells growing on a petri dish or other solid or semisolid support are exposed to.

The cell culture media used for culture expansion contains all essential amino acids and may also contain non-essential amino acids. In general, amino acids are classified into essential amino acids (Thr, Met, Val, Leu, Ile, Phe, Trp, Lys, His) and non-essential amino acids (Gly, Ala, Ser, Cys, Gln, Asn, Asp, Tyr, Arg, Pro).

Those of skill in the art will appreciate that for optimal results, the basal medium must be appropriate for the cell line of interest. For example, it may be necessary to increase the level of glucose (or other energy source) in the basal medium, or to add glucose (or other energy source) during the course of culture, if this energy source is found to be depleted and to thus limit growth. In an example, dissolved oxygen (DO) levels can also be controlled.

In an example, the cell culture medium contains human derived additives. For example, human serum and human platelet cell lysate can be added to the cell culture media.

In an example, the cell culture medium contains only human derived additives. Thus, in an example, the cell culture media is xeno-free. For avoidance of doubt, in these examples, the culture medium is free of animal proteins. In an example, cell culture medium used in the methods of the disclosure is free of animal components.

In an example, the culture medium comprises serum. In other examples the culture medium is fetal bovine serum free culture medium comprising growth factors that promote mesenchymal lineage precursor or stem cell proliferation. In an embodiment, the culture medium is serum free stem cell culture medium. In an example, the cell culture medium comprises:
a basal medium;
platelet derived growth factor (PDGF);
fibroblast growth factor 2 (FGF2).

In an example, the culture medium comprises platelet derived growth factor (PDGF) and fibroblast growth factor 2 (FGF2), wherein the level of FGF2 is less than about 6 ng/ml. For example, the FGF2 level may be less than about 5 ng/ml, less than about 4 ng/ml, less than about 3 ng/ml, less than about 2 ng/ml, less than about 1 ng/ml. In other examples, the FGF2 level is less than about 0.9 ng/ml, less than about 0.8 ng/ml, less than about 0.7 ng/ml, less than about 0.6 ng/ml, less than about 0.5 ng/ml, less than about 0.4 ng/ml, less than about 0.3 ng/ml, less than about 0.2 ng/ml.

In another example, the level of FGF2 is between about 1 pg/ml and 100 pg/ml. In another example, the level of FGF2 is between about 5 pg/ml and 80 pg/ml.

In an example, the PDGF is PDGF-BB. In an example, the level of PDGF-BB is between about 1 ng/ml and 150 ng/ml. In another example, the level of PDGF-BB is between about 7.5 ng/ml and 120 ng/ml. In another example, the level of PDGF-BB is between about 15 ng/ml and 60 ng/ml. In another example, the level of PDGF-BB is at least about 10 ng/ml. In another example, the level of PDGF-BB is at least about 15 ng/ml. In another example, the level of PDGF-BB is at least about 20 ng/ml. In another example, the level of PDGF-BB is at least about 21 ng/ml. In another example, the level of PDGF-BB is at least about 22 ng/ml. In another example, the level of PDGF-BB is at least about 23 ng/ml. In another example, the level of PDGF-BB is at least about 24 ng/ml. In another example, the level of PDGF-BB is at least about 25 ng/ml.

In another example, the PDGF is PDGF-AB. In an example, the level of PDGF-AB is between about 1 ng/ml and 150 ng/ml. In another example, the level of PDGF-AB is between about 7.5 ng/ml and 120 ng/ml. In another example, the level of PDGF-AB is between about 15 ng/ml and 60 ng/ml. In another example, the level of PDGF-AB is at least about 10 ng/ml. In another example, the level of PDGF-AB is at least about 15 ng/ml. In another example, the level of PDGF-AB is at least about 20 ng/ml. In another example, the level of PDGF-AB is at least about 21 ng/ml. In another example, the level of PDGF-AB is at least about 22 ng/ml. In another example, the level of PDGF-AB is at least about 23 ng/ml. In another example, the level of PDGF-AB is at least about 24 ng/ml. In another example, the level of PDGF-AB is at least about 25 ng/ml.

In other examples, additional factors can be added to the cell culture medium. In an example, the culture medium further comprising EGF. EGF is a growth factor that stimulates cell proliferation by binding to its receptor EGFR. In an example, the method of the present disclosure comprises culturing a population of stem cells in a fetal bovine serum free cell culture medium further comprising EGF. In an example, the level of EGF is between about 0.1 and 7 ng/ml. For example, the level of EGF can be at least about 5 ng/ml.

In another example, the level of EGF is between about 0.2 ng/ml and 3.2 ng/ml. In another example, the level of EGF is between about 0.4 ng/ml and 1.6 ng/ml. In another example, the level of EGF is between about 0.2 ng/ml. In another example, the level of EGF is at least about 0.3 ng/ml. In another example, the level of EGF is at least about 0.4 ng/ml. In another example, the level of EGF is at least about 0.5 ng/ml. In another example, the level of EGF is at least about 0.6 ng/ml. In another example, the level of EGF is at least about 0.7 ng/ml. In another example, the level of EGF is at least about 0.8 ng/ml. In another example, the level of EGF is at least about 0.9 ng/ml. In another example, the level of EGF is at least about 1.0 ng/ml.

In the above examples, basal medium such as Alpha MEM or StemSpan™ can be supplemented with the referenced quantity of growth factor. In an example, the culture medium comprises Alpha MEM or StemSpan™ supplemented with 32 ng/ml PDGF-BB, 0.8 ng/ml EGF and 0.02 ng/ml FGF.

In other examples, additional factors can be added to the cell culture medium. For example, the cell culture media can be supplemented with one or more stimulatory factors selected from the group consisting of epidermal growth factor (EGF), 1α,25-dihydroxyvitamin D3 (1,25D), tumor necrosis factor α (TNF-α), interleukin-1β (IL-1β) and stromal derived factor 1α (SDF-1α). In another embodiment, cells may also be cultured in the presence of at least one cytokine in an amount adequate to support growth of the cells. In another embodiment, cells can be cultured in the presence of heparin or a derivative thereof. For example, the cell culture medium may contain about 50 ng/ml of heparin. In other examples, the cell culture medium contains about 60 ng/ml of heparin, about 70 ng/ml of heparin, about 80 ng/ml of heparin, about 90 ng/ml of heparin, about 100 ng/ml of heparin, about 110 ng/ml of heparin, about 110 ng/ml of heparin, about 120 ng/ml of heparin, about 130 ng/ml of heparin, about 140 ng/ml of heparin, about 150 ng/ml of heparin or a derivative thereof. In an example, the heparin derivative is a sulphate). Various forms of heparin sulphate are known in the art and include heparin sulphate 2 (HS2). HS2 can be derived from various sources including for example, the liver of male and/or female mammals. Thus, an exemplary heparin sulphate includes male liver heparin sulphate (MML HS) and female liver heparin sulphate (FML HS).

In another example, the cell culture medium of the present disclosure promotes stem cell proliferation while maintaining stem cells in an undifferentiated state. Stem cells are considered to be undifferentiated when they have not committed to a specific differentiation lineage. As discussed above, stem cells display morphological characteristics that distinguish them from differentiated cells. Furthermore, undifferentiated stem cells express genes that may be used as markers to detect differentiation status. The polypeptide products may also be used as markers to detect differentiation status. Accordingly, one of skill in the art could readily determine whether the methods of the present disclosure maintain stem cells in an undifferentiated state using routine morphological, genetic and/or proteomic analysis.

### Modification of the Cells

The mesenchymal lineage precursor or stem cells disclosed herein may be altered in such a way that upon administration, lysis of the cell is inhibited. Alteration of an antigen can induce immunological non-responsiveness or tolerance, thereby preventing the induction of the effector phases of an immune response (e.g., cytotoxic T cell generation, antibody production etc.) which are ultimately responsible for rejection of foreign cells in a normal immune response. Antigens that can be altered to achieve this goal include, for example, MHC class I antigens, MHC class II antigens, LFA-3 and ICAM-1.

The mesenchymal lineage precursor or stem cells may also be genetically modified to express proteins of importance for the differentiation and/or maintenance of striated skeletal muscle cells. Exemplary proteins include growth factors (TGF-β, insulin-like growth factor 1 (IGF-1), FGF), myogenic factors (e.g. myoD, myogenin, myogenic factor 5 (Myf5), myogenic regulatory factor (MRF)), transcription factors (e.g. GATA-4), cytokines (e.g. cardiotropin-1), members of the neuregulin family (e.g. neuregulin 1, 2 and 3) and homeobox genes (e.g. Csx, tinman and NKx family).

### Compositions

In an example, the present disclosure encompasses an opioid sparing composition comprising mesenchymal lineage precursor or stem cells (MLPSCs) and hyaluronic acid (HA). In an example, the composition is administered and opioids are subsequently spared after administration. For example, opioids can be spared after 3 months. In another example, opioids can be spared after 6 months. In another example, opioids can be spared after 12 months. In another example, opioids can be spared after 18 months. In another example, opioids can be spared after 24 months. In another example, opioids can be spared at 36 months. In another example, opioids can be stopped after 18 months. In another example, opioids can be stopped at 36 months.

In an example, the opioid sparing composition comprises 1% HA. In another example, the opioid sparing composition is administered to subjects that are using an opioid. For example, the subject can be using an opioid for the treatment of pain.

Mesenchymal lineage or stem cells disclosed herein can be culture expanded from a cryopreserved intermediate to produce a preparation containing at least one therapeutic dose. In an example, the methods of the present disclosure encompass administration of one dose. In other examples, more than one dose is administered.

In an example, compositions of the disclosure comprise between 10×10⁶ cells and 35×10⁶ cells. In another example, the composition comprises between 20×10⁶ cells and 30×10⁶ cells. In other examples, the composition comprises at least 5 million cells. In another example, the composition comprises 6 million cells.

In one example, compositions of the disclose comprise a pharmaceutically acceptable carrier and/or excipient. The terms “carrier” and “excipient” refer to compositions of matter that are conventionally used in the art to facilitate the storage, administration, and/or the biological activity of an active compound (see, e.g., Remington's Pharmaceutical Sciences, 16th Ed., Mac Publishing Company (1980). A carrier may also reduce any undesirable side effects of the active compound. A suitable carrier is, for example, stable, e.g., incapable of reacting with other ingredients in the carrier. In one example, the carrier does not produce significant local or systemic adverse effect in recipients at the dosages and concentrations employed for treatment.

Suitable carriers for the present disclosure include those conventionally used, e.g., water, saline, aqueous dextrose, lactose, Ringer's solution, a buffered solution, hyaluronan and glycols are exemplary liquid carriers, particularly (when isotonic) for solutions. Suitable pharmaceutical carriers and excipients include starch, cellulose, glucose, lactose, sucrose, gelatin, malt, rice, flour, chalk, silica gel, magnesium stearate, sodium stearate, glycerol monostearate, sodium chloride, glycerol, propylene glycol, water, ethanol, and the like.

In another example, a carrier is a media composition, e.g., in which a cell is grown or suspended. Such a media composition does not induce any adverse effects in a subject to whom it is administered. Exemplary carriers and excipients do not adversely affect the viability of a cell and/or the ability of a cell to treat or prevent disease.

In one example, the carrier or excipient provides a buffering activity to maintain the cells and/or soluble factors at a suitable pH to thereby exert a biological activity, e.g., the carrier or excipient is phosphate buffered saline (PBS). PBS represents an attractive carrier or excipient because it interacts with cells and factors minimally and permits rapid release of the cells and factors, in such a case, the composition of the disclosure may be produced as a liquid for direct application to the blood stream or into a tissue or a region surrounding or adjacent to a tissue, e.g., by injection.

Compositions of the disclosure may be cryopreserved. Cryopreservation of mesenchymal lineage precursor or stem cells can be carried out using slow-rate cooling methods or ‘fast’ freezing protocols known in the art. Preferably, the method of cryopreservation maintains similar phenotypes, cell surface markers and growth rates of cryopreserved cells in comparison with unfrozen cells.

The cryopreserved composition may comprise a cryopreservation solution. The pH of the cryopreservation solution is typically 6.5 to 8, preferably 7.4.

The cyropreservation solution may comprise a sterile, non-pyrogenic isotonic solution such as, for example, PlasmaLyte ATM. 100 mL of PlasmaLyte ATM contains 526 mg of sodium chloride, USP (NaCl); 502 mg of sodium gluconate (C6H11NaO7); 368 mg of sodium acetate trihydrate, USP (C2H3NaO2·3H2O); 37 mg of potassium chloride, USP (KCl); and 30 mg of magnesium chloride, USP (MgCl2·6H2O). It contains no antimicrobial agents. The pH is adjusted with sodium hydroxide. The pH is 7.4 (6.5 to 8.0).

The cryopreservation solution may comprise Profreeze™. The cryopreservation solution may additionally or alternatively comprise culture medium, for example, αMEM.

To facilitate freezing, a cryoprotectant such as, for example, dimethylsulfoxide (DMSO), is usually added to the cryopreservation solution. Ideally, the cryoprotectant should be nontoxic for cells and patients, nonantigenic, chemically inert, provide high survival rate after thawing and allow transplantation without washing. However, the most commonly used cryoprotector, DMSO, shows some cytotoxicity. Hydroxylethyl starch (HES) may be used as a substitute or in combination with DMSO to reduce cytotoxicity of the cryopreservation solution.

The cryopreservation solution may comprise one or more of DMSO, hydroxyethyl starch, human serum components and other protein bulking agents. In one example, the cryopreserved solution comprises Plasma-Lyte A (70%), DMSO (10%), HSA (25%) solution, the HSA solution comprising 5% HSA and 15% buffer.

In an example, the cryopreservation solution may further comprise one or more of methylcellulose, polyvinyl pyrrolidone (PVP) and trehalose.

The cryopreserved composition may be thawed and administered directly to the subject or added to another solution, for example, comprising hyaluronic acid. Alternatively, the cryopreserved composition may be thawed and the mesenchymal lineage precursor or stem cells resuspended in an alternate carrier prior to administration.

The compositions described herein may be administered alone or as admixtures with other cells. The cells of different types may be admixed with a composition of the disclosure immediately or shortly prior to administration, or they may be co-cultured together for a period of time prior to administration.

The exact amount of cells to be administered is dependent upon a variety of factors, including the age, weight, and sex of the subject, and the extent and severity of the disorder being treated.

Despite the number of cells provided in the composition, in an example, 50×10⁶ to 200×10⁷ cells are administered. In other examples, 60×10⁶ to 200×10⁶ cells or 75×10⁶ to 150×10⁶ cells are administered. In an example, 75×10⁶ cells are administered. In another example, 150×10⁶ cells are administered. In these examples cells can be administered as a single does or over multiple doses.

In an example, the composition comprises greater than 5.000×10⁶ viable cells/mL. In another example, the composition comprises greater than 5.50×10⁶ viable cells/mL. In another example, the composition comprises greater than 6.00×10⁶ viable cells/mL. In another example, the composition comprises greater than 6.50×10⁶ viable cells/mL. In another example, the composition comprises greater than 6.68×10⁶ viable cells/mL.

In an example, the mesenchymal lineage precursor or stem cells comprise at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99% of the cell population of the composition.

In an example, the composition may optionally be packaged in a suitable container with written instructions for a desired purpose.

Compositions of the disclosure may be administered systemically, such as, for example, by intravenous administration. In an example, compositions are administered into a painful intervertebral disc. In an example, compositions of the disclosure are administered into the nucleus pulposus or the annulus fibrosis of an intervertebral disc.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed and/or referenced herein are incorporated herein in their entirety.

This application claims priority from AU2021900147 filed on 22 Jan. 2021, AU2021900250 filed on 4 Feb. 2021 and U.S. 63/298,391 filed on 11 Jan. 2022, the disclosures of which are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### Examples

### Compositions

Treatment compositions comprise human bone marrow-derived allogeneic MIPCs, expanded ex vivo, and cryopreserved. Cells are in composition alone (MIPC) or with 1% hyaluronic acid (HA) (MMPC+HA).

### Patients

Subjects with chronic low back pain (>6 months) associated with moderate radiographic degenerative changes of a disc.

### Baseline Data

| | | MPC + | | All |
|---|---|---|---|---|
| Characteristic | MPC | HA | Placebo | Subjects |
| Age (mean) | 42.1 | 42.9 | 43.3 | 42.8 |
| Gender | | | | |
| Male | 58.0% | 58.1% | 53.8% | 56.7% |
| Female | 42.0% | 41.9% | 46.2% | 43.3% |
| Smoking Status | | | | |
| Never Smoked | 55.2% | 62.8% | 64.4% | 60.6% |
| Current Smoker | 16.8% | 15.5% | 16.7% | 16.3% |
| Previous Smoker | 28.0% | 21.7% | 18.9% | 23.0% |
| BMI | | | | |
| Mean | 27.6 | 27.5 | 27.0 | 27.4 |
| BMI <30 | 73.4% | 70.5% | 75.8% | 73.3% |
| BMI ≥30 | 26.6% | 29.5% | 24.2% | 26.7% |
| Duration of Low Back Pain | | | | |
| Mean | 106.8 | 100.9 | 95.4 | 101.2 |
| Median | 78.5 | 67.9 | 65.0 | 68.8 |
| Duration Since DDD | | | | |
| Diagnosis | | | | |
| Mean | 48.3 | 40.2 | 46.2 | 45.0 |
| Median | 19.9 | 23.5 | 20.7 | 22.1 |
| Opioid Use at Baseline | | | | |
| (Derived from E-Diary) | | | | |
| Opioid Users | 44.1% | 41.9% | 38.6% | 41.6% |
| Opioid Non-Users | 53.1% | 57.4% | 59.8% | 56.7% |
| Missing | 2.8% | 0.8% | 1.5% | 1.7% |
| Treated Level Pfirrmann | | | | |
| Score | | | | |
| Grade II | 0.0% | 0.0% | 0.8% | 0.2% |
| Grade III | 13.3% | 18.6% | 17.4% | 16.3% |
| Grade IV | 51.7% | 58.9% | 52.3% | 54.2% |
| Grade V | 18.9% | 10.9% | 16.7% | 15.6% |
| Grade VI | 15.4% | 11.6% | 12.9% | 13.4% |
| Grade VII | 0.7% | 0.0% | 0.0% | 0.2% |
| Number of Degenerated | | | | |
| Lumbar Discs (Pfirrmann | | | | |
| Grade III or higher) | | | | |
| 0 Discs | 0.0% | 0.0% | 0.8% | 0.2% |
| 1 Disc | 21.0% | 20.2% | 14.4% | 18.6% |
| 2 Discs | 15.4% | 17.1% | 14.4% | 15.6% |
| 3 Discs | 11.2% | 9.3% | 13.6% | 11.4% |
| 4 Discs | 7.7% | 3.1% | 1.5% | 4.2% |
| 5 Discs | 44.8% | 50.4% | 55.3% | 50.0% |
| Provocative Discography | | | | |
| at Screening | | | | |
| Not Performed | 29.4% | 20.9% | 24.2% | 25.0% |
| Performed in Target | 37.8% | 48.1% | 47.0% | 44.1% |
| and Control Level(s) | | | | |
| Only Performed in | 32.2% | 31.0% | 28.8% | 30.7% |
| Target Level | | | | |
| Only Performed in | 0.7% | 0.0% | 0.0% | 0.2% |
| Control Level(s) | | | | |
| Herniation Score for | | | | |
| Index Level | | | | |
| None | 13.3% | 14.7% | 17.4% | 15.1% |
| Bulge | 67.1% | 66.7% | 68.9% | 67.6% |
| Protrusion | 18.9% | 17.8% | 12.9% | 16.6% |
| Extrusion | 0.7% | 0.8% | 0.8% | 0.7% |
| Protocol Version | | | | |
| Version 4.0 or earlier | 21.7% | 17.1% | 20.5% | 19.8% |
| Version 5.0 or later | 78.3% | 82.9% | 79.5% | 80.2% |
| Low Back Pain VAS - | | | | |
| Average Pain Over | | | | |
| 24 Hours | | | | |
| Mean | 60.3 | 60.4 | 57.1 | 59.3 |
| Median | 59.0 | 59.0 | 55.0 | 58.0 |
| Low Back Pain VAS - | | | | |
| Average Pain Over | | | | |
| 24 hours | | | | |
| <40 mm | 1.4% | 0.0% | 2.3% | 1.2% |
| 40-60 mm | 52.4% | 55.8% | 56.8% | 55.0% |
| 61-80 mm | 39.2% | 35.7% | 34.1% | 36.4% |
| 81-90 mm | 6.3% | 7.0% | 6.1% | 6.4% |
| >90 mm | 0.0% | 0.0% | 0.0% | 0.0 |
| Missing | 0.7% | 1.6% | 0.8% | 1.0% |
| Oswestry Disability | | | | |
| Index (ODI) Score | | | | |
| Mean | 41.7 | 41.3 | 42.2 | 41.7 |
| Median | 40.0 | 40.0 | 40.0 | 40.0 |
| ODI Score | | | | |
| <30% | 0.0% | 1.6% | 0.8% | 0.7% |
| 30-40% | 55.2% | 52.7% | 54.5% | 54.2% |
| 41-60% | 39.2% | 40.3% | 39.4% | 39.6% |
| 61-80% | 5.6% | 4.7% | 5.3% | 5.2% |
| 81-90% | 0.0% | 0.8% | 0.0% | 0.2% |
| >90% | 0.0% | 0.0% | 0.0% | 0.0% |
| EQ5D VAS Score | | | | |
| Mean | 66.1 | 68.3 | 68.7 | 67.7 |
| Median | 70.0 | 70.0 | 70.0 | 70.0 |
| EQ5D Index Score | | | | |
| Mean | 0.6623 | 0.6694 | 0.6752 | 0.6688 |
| Median | 0.6890 | 0.7040 | 0.6780 | 0.6890 |

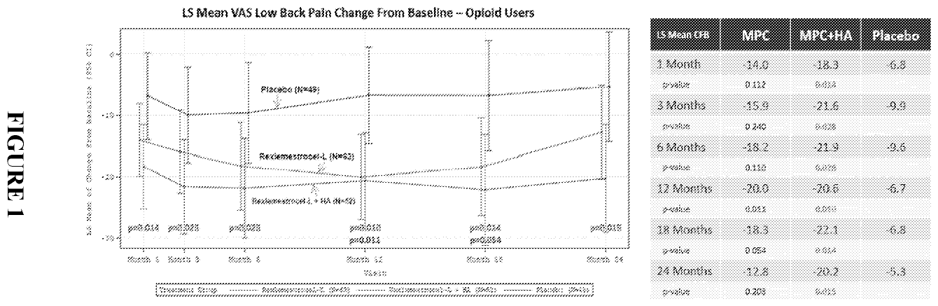

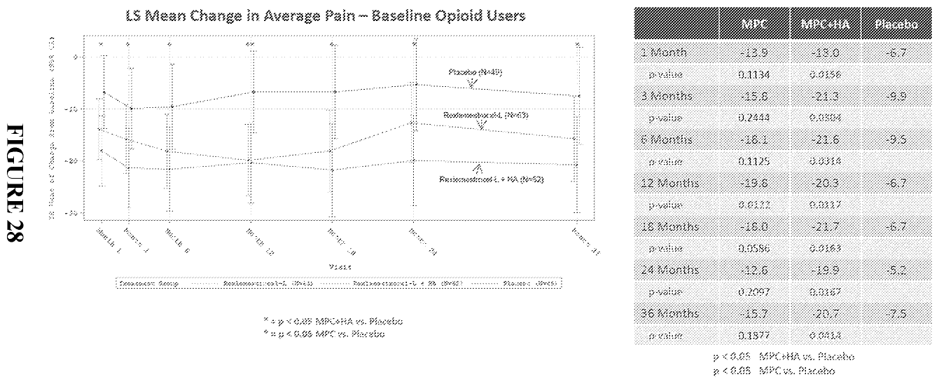

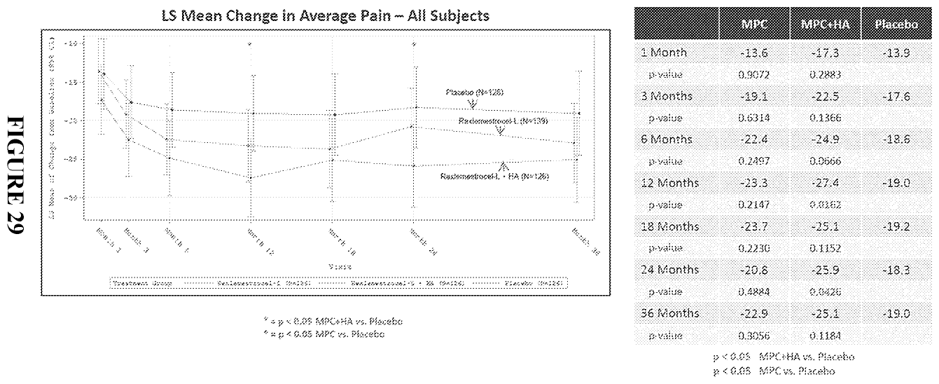

Both treatment with MIPC and MPC+HA reduced mean VAS Low Back pain in all patients using opioids relative to baseline. The most pronounced reduction was observed in subjects administered MIPC+HA. Indeed, MPC+HA had significantly greater mean pain reduction from baseline at every time point over 36 months compared with placebo (FIGS. 1 and 28). Looking more broadly (FIG. 29), treatment with MPC+HA reduced mean VAS Low Back pain in all patients relative to baseline at 12 (p=0.0162) and 24 months (p=0.0426).

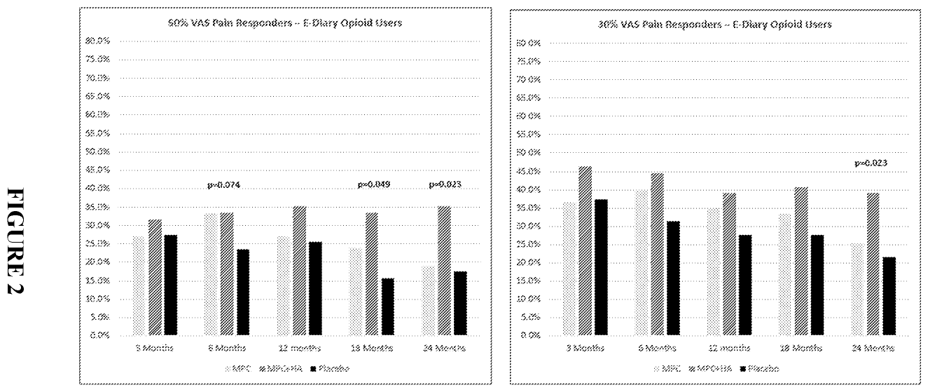

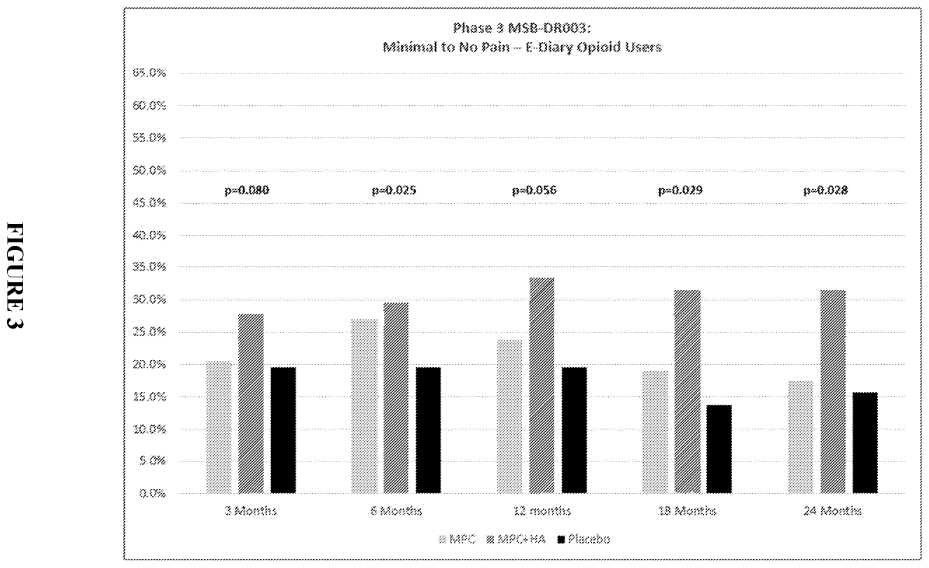

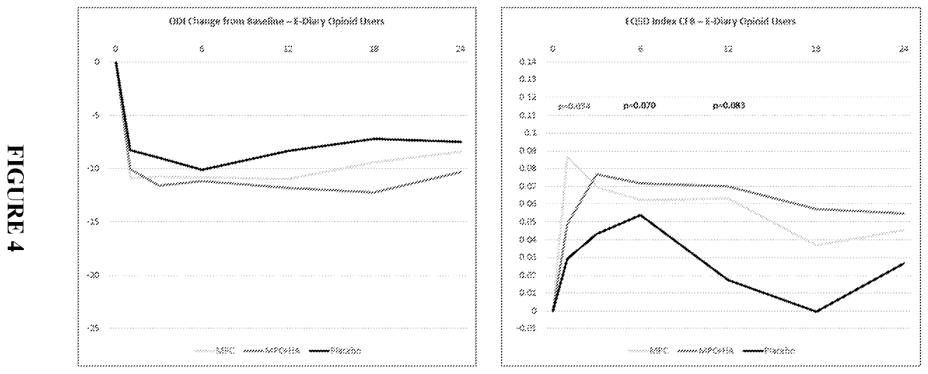

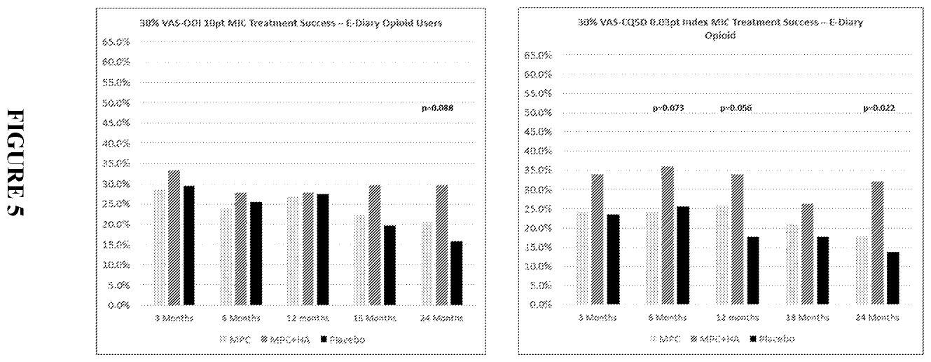

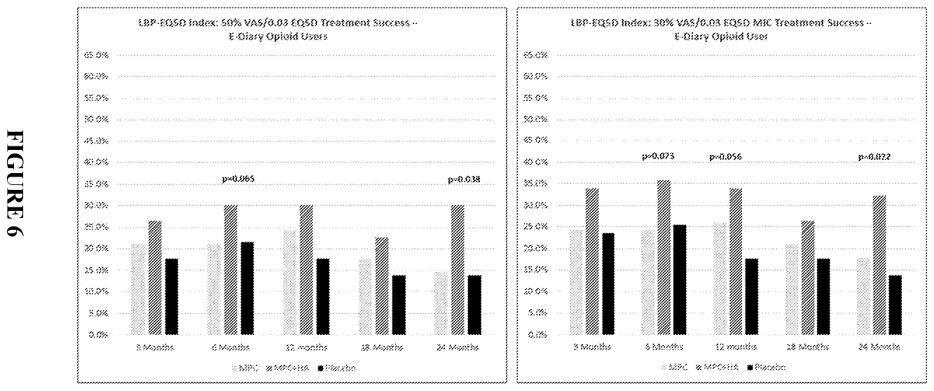

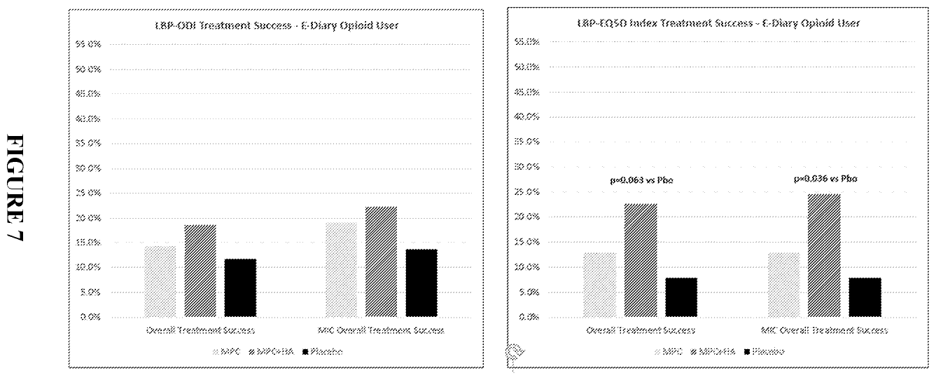

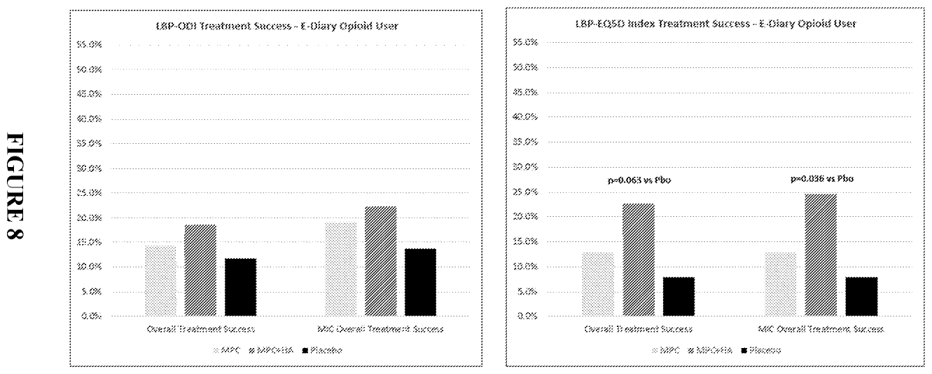

Despite instructions not to change treatment regimens, opioid using patients treated with MPC+HA showed approximately 40% reduction in opioid use over 24 months. In these patients, MPC+HA significantly:
reduced mean VAS scores (FIG. 1);
increased proportion of 50% and 30% VAS responders (FIG. 2);
increased proportion of patients with minimal/no pain at 12 and 24 months (FIG. 3). Pain Responders defined as subjects with a 50% reduction in Low Back Pain VAS score from baseline AND no post-treatment intervention through time point being evaluated;
increased mean EQ-5D scores (FIGS. 4 and 5); and,
increased proportion of patients achieving composite of treatment success in pain and function through 24 months (FIGS. 6 to 8). Regarding LBP-ODI treatment success, overall success defined as subject with a 50% reduction in LBP VAS score from baseline AND 15-point improvement in ODI score from baseline at both 12 & 24 months AND no post-treatment intervention through 24 months. For MIC, success defined as subjects with a 30% reduction in LBP VAS score from baseline AND 10-point improvement in ODI score from baseline at both 12 & 24 months AND no post-treatment intervention through 24 months. For LBP-EQ5D treatment success defined as subjects at both 12 and 24 months with 0.03 improvement in EQ5D and either 50% reduction in VAS (overall) or 30% reduction in VAS (MIC) and no intervention.

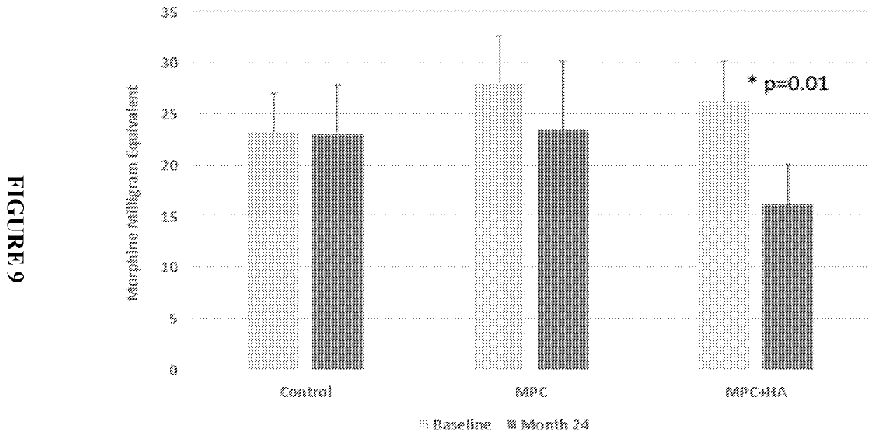

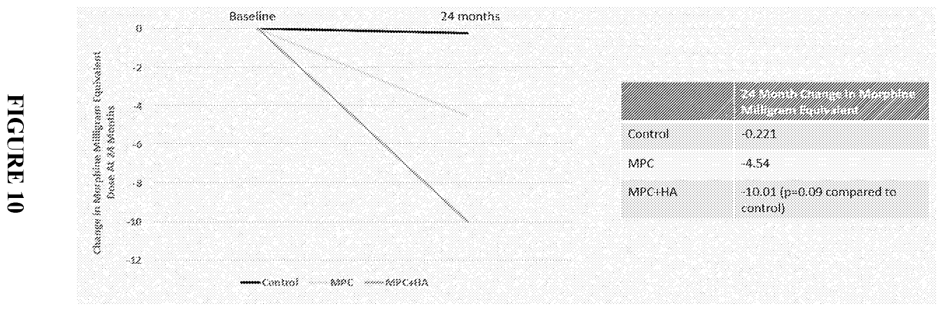

Notably, both MPC and MPC+HA reduced morphine milligram equivalents over 24 months in opioid users (FIGS. 9 and 10) with a significant reduction being observed with MPC+HA (p=0.01).

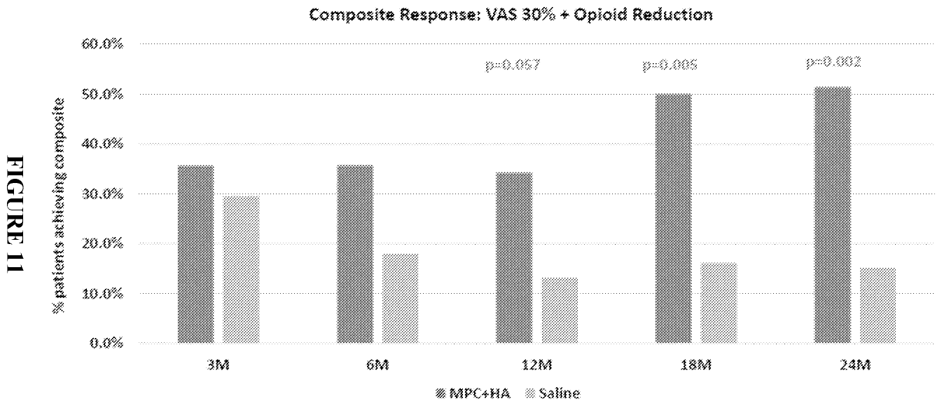

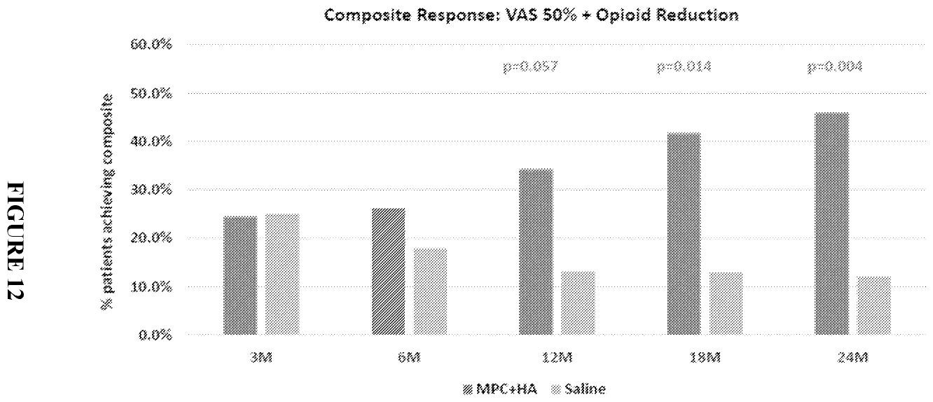

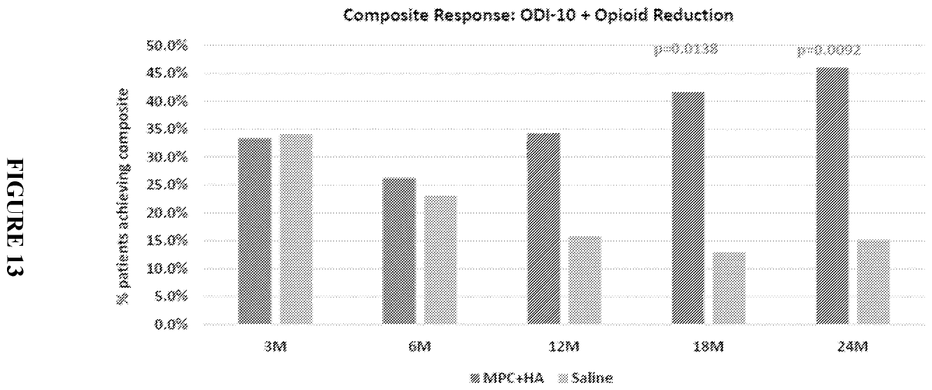

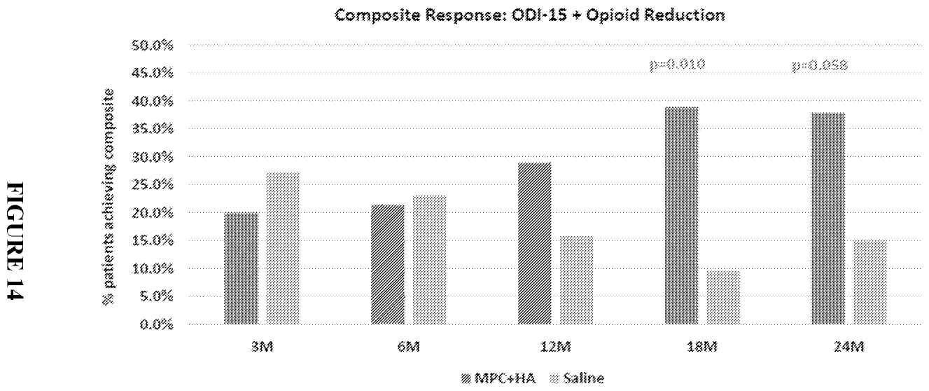

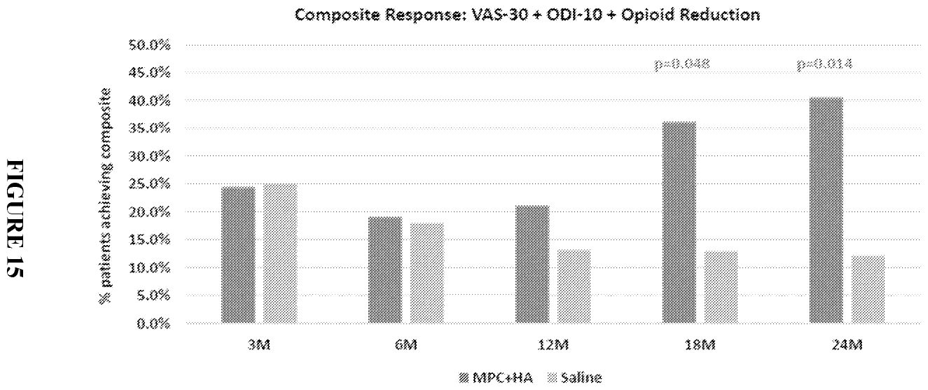

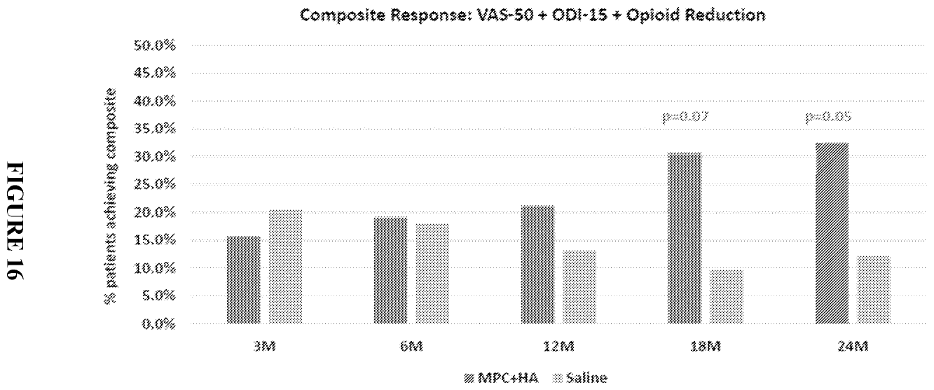

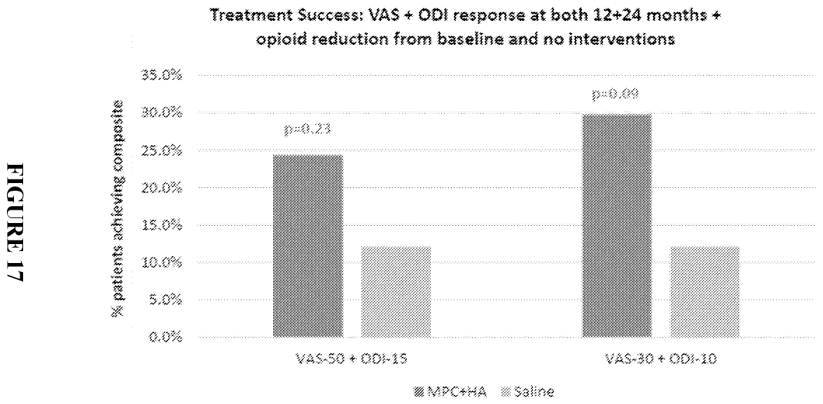

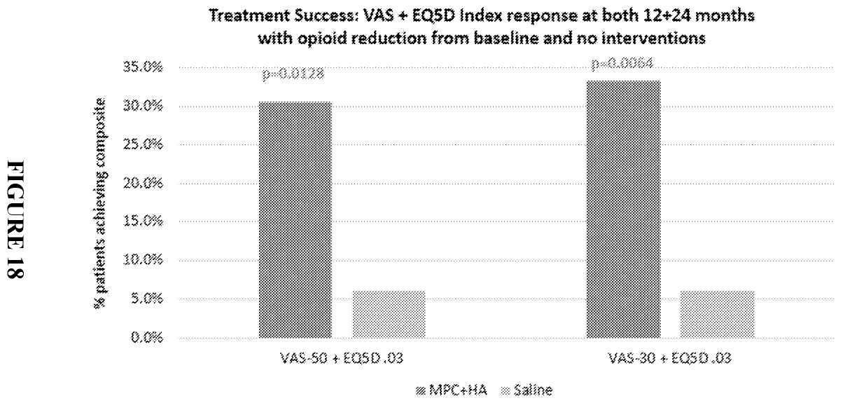

Further analysis of treatment response in patients administered MPC+HA revealed:
Increased proportion of opioid users achieving both VAS 30% and VAS 50% pain response over 24 Months while reducing Daily Opioid Use (FIGS. 11 and 12);
Increased proportion of opioid users achieving both ODI 10-point function response (FIG. 13), 15-point function response (FIG. 14), VAS 30%+ODI 10-point function response (FIG. 15) and, VAS 50%+ODI 15-point function response (FIG. 16) over 24 months while also reducing daily opioid use; and,
Increased proportion of opioid users achieving pain/function composite responses at both 12 and 24 months while also reducing opioid use (FIGS. 17 and 18).

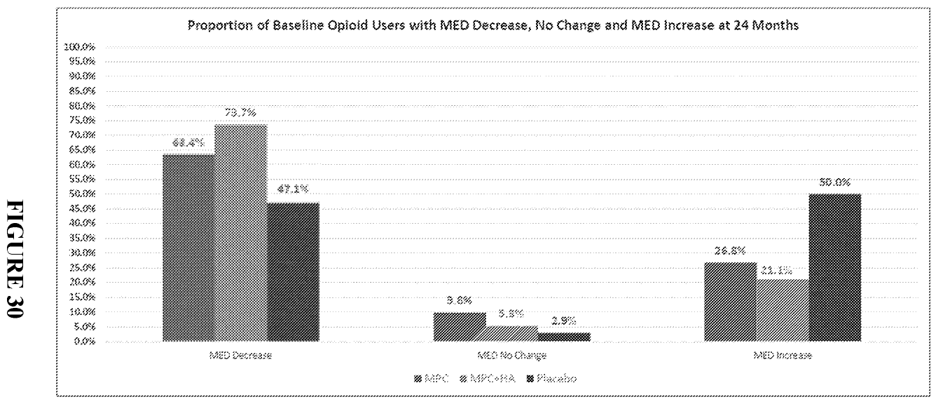

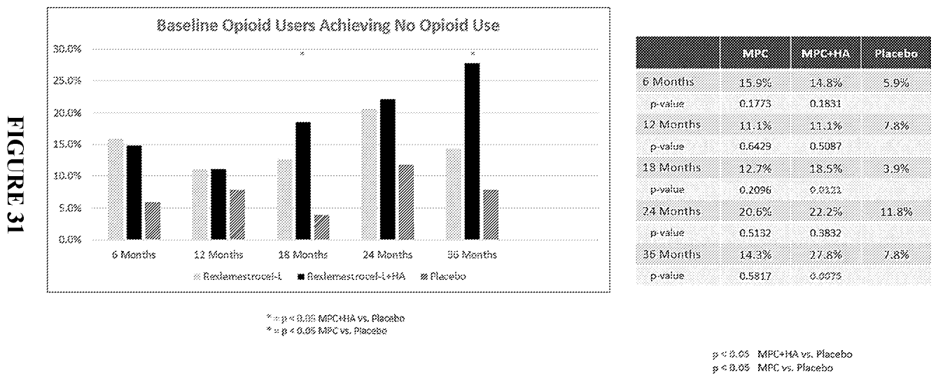

Taken together with the above findings, these data indicate that compositions comprising MLPSCs may be useful as opioid sparing agents and, in particular in the context of subjects with chronic pain such as lower back pain. This is a significant finding in view of the sustained opioid use in this patient population and the ongoing side effects of the same. Analysis of opioid use 24 months following administration of cell therapy provided further evidence that compositions comprising MLPSCs may be useful as opioid sparing agents. 73.7% and 63.4% of patients receiving MPC+HA and MPC respectively decreased their pain medication usage. In contrast, only 50% of placebo patients decreased their pain medication usage (FIG. 30). Notably, a significant increase in patients achieving no opioid use was observed at 18 months (p=0.0121) and 36 months (p=0.0075) following administration of MPC+HA (FIG. 31). The durable elimination of opioid use is remarkable and, in view of the chronic nature of pathology in these patients, represents a particularly surprising result.

### Early Intervention

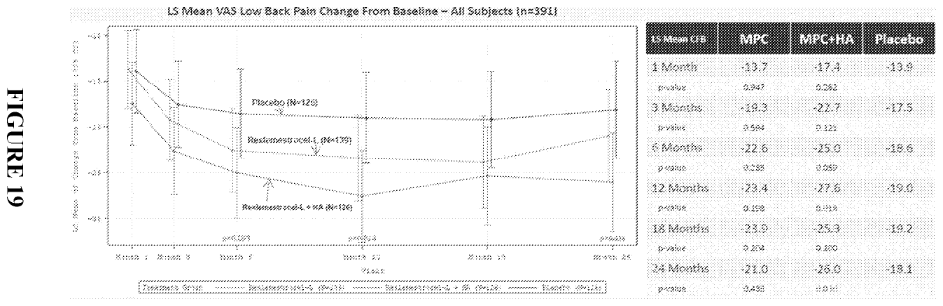

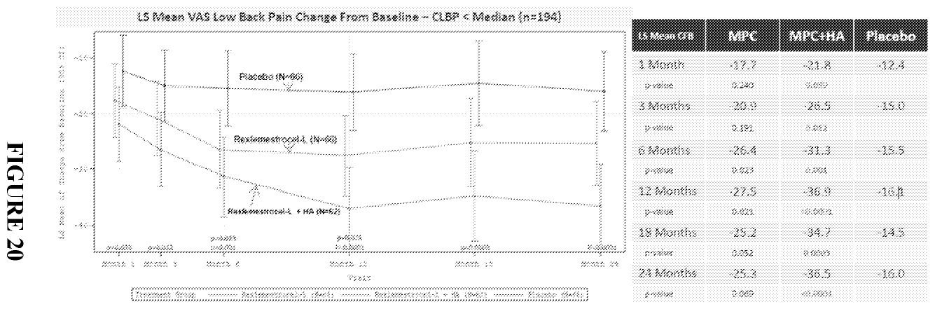

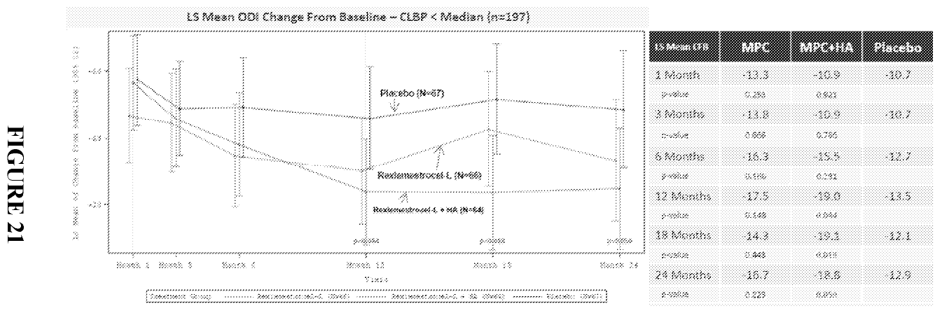

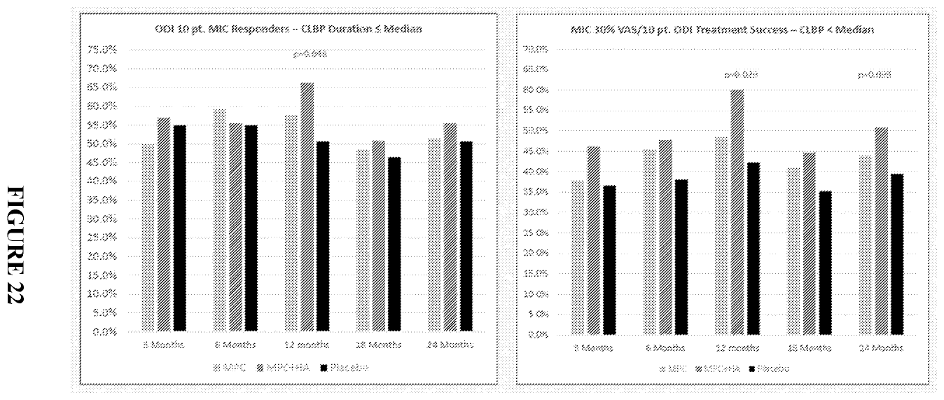

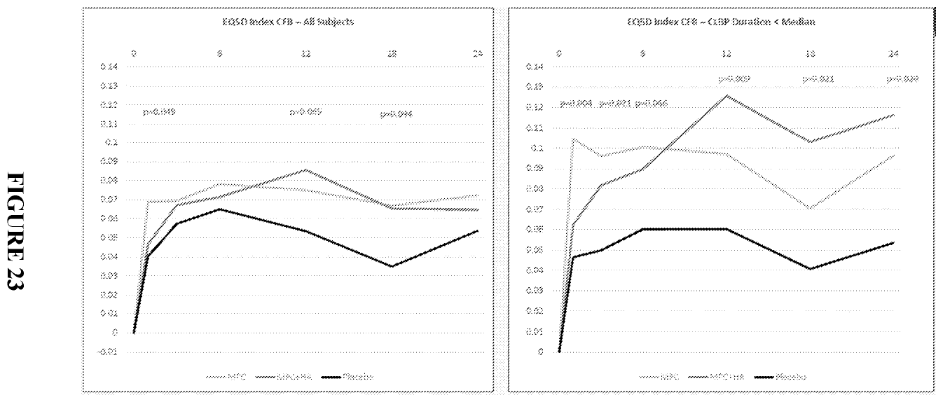

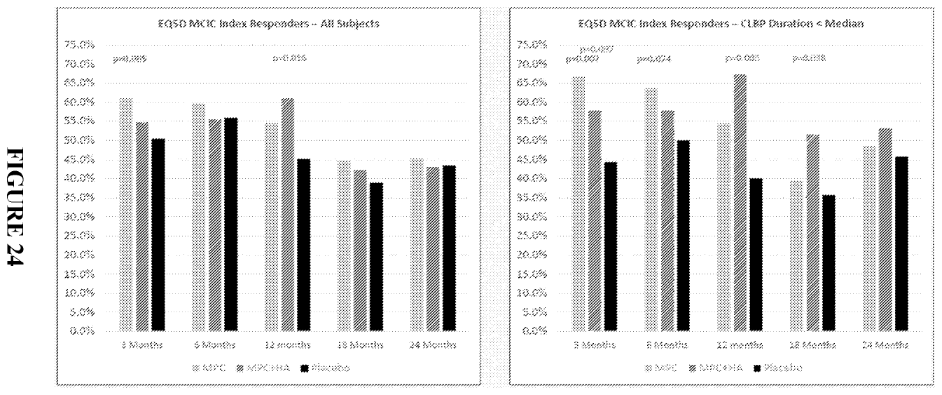

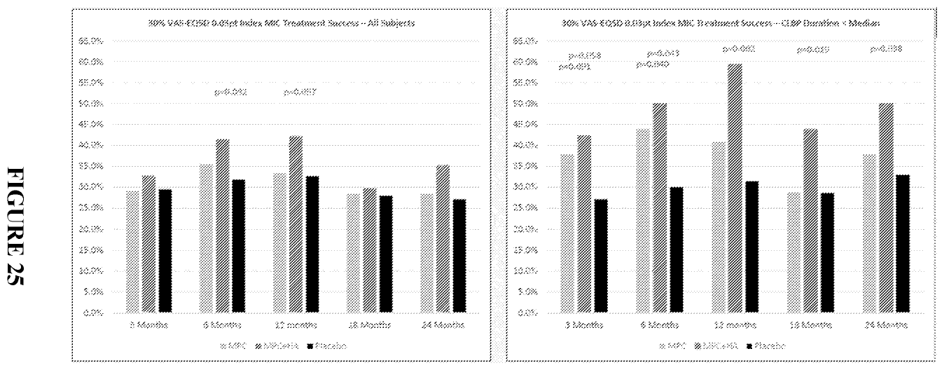

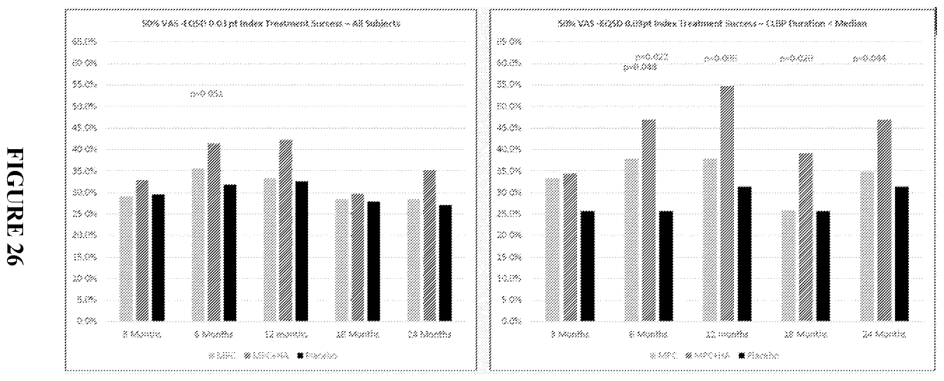

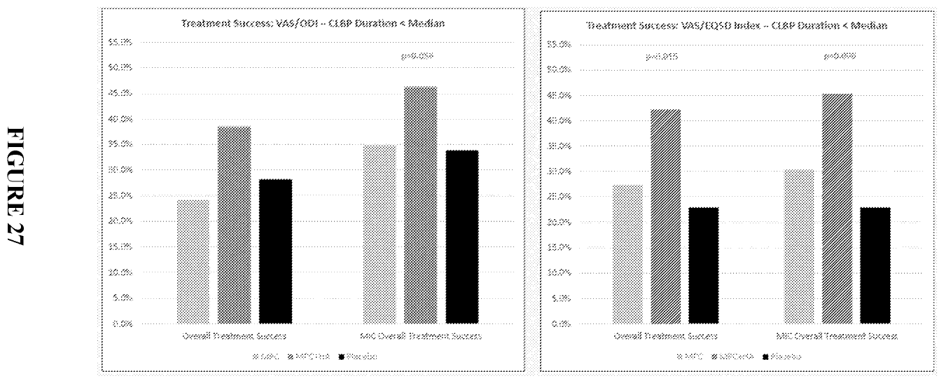

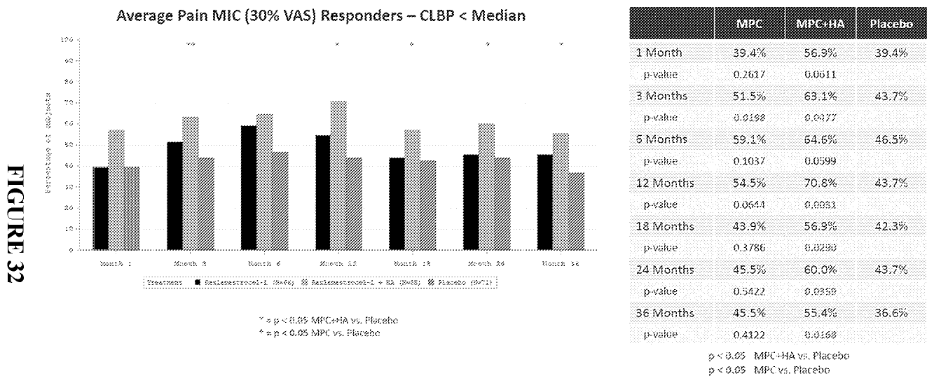

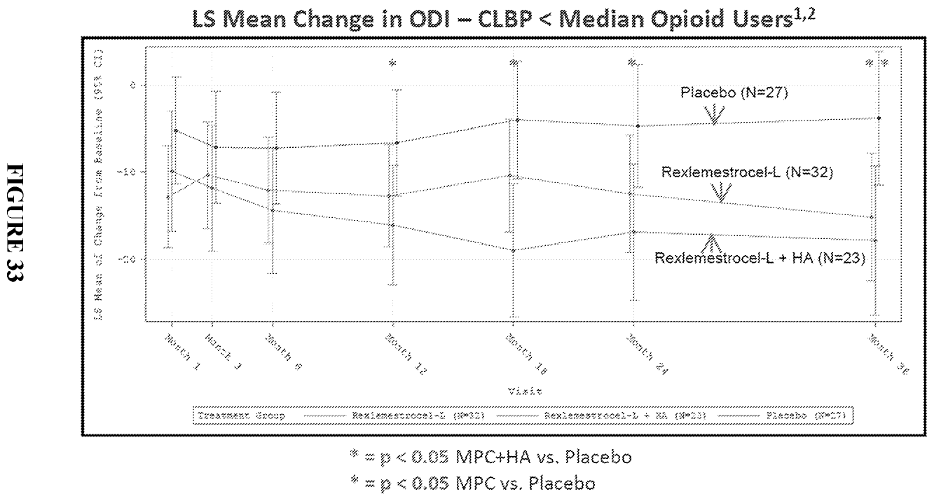

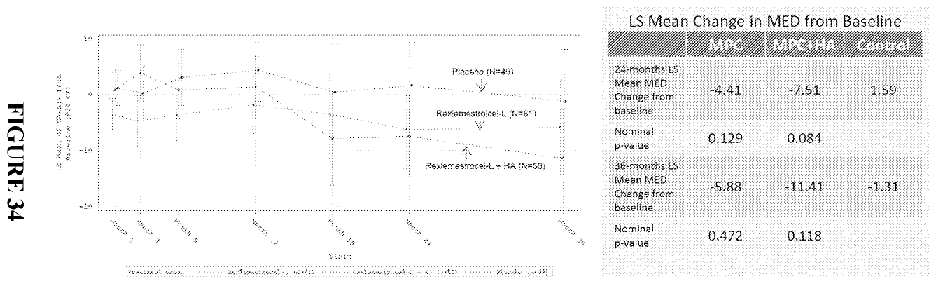

A single dose of MPC treatment reduced pain in all patients through 36 months (FIGS. 19 and 32). The median chronic lower back pain (CLBP) duration for these patients was 68 months. Further analysis of the data from patients with CLBP less than 68 months revealed that MPC treatment effect on pain (both MPC and MPC+HA) was most evident in these patients (FIG. 20; FIG. 21). Notably, treatment effect on ODI function score was most evident in patients with CLBP duration less than 68 months (FIG. 21), in particular in those patients that were also opioid users (FIG. 33). Further, MPC treatment (both MPC and MPC+HA) reduced morphine equivalent dose over 36 months in opioid users (FIG. 34). It was also notable that administration of MPC+HA significantly increased the number of composite responders (30% VAS/0.03EQ5D) at 12 months (FIG. 22; trends towards an increase in the number of composite responders at 12 months were also observed in the MPC treatment group). Improved EQ5D was noted in patients with CLBP duration less than 68 months following both MPC and MPC+HA treatment (FIGS. 23-26; RHS shows <68 months CLBP). These data are particularly promising because EQ5D may more effectively assess treatment response in earlier stages of CLBP. In addition, MPC+HA administration increased treatment success for pain/function (VAS/ODI) and pain/quality of life (VAS/EQ5D index) composites at both 12 and 24 months (FIG. 27).

The most marked improvement in patients with CLBP for less than 68 months was noted following administration of MPC+HA which significantly:
reduced mean VAS scores;
increased proportion of 50% and 30% VAS responders;
increased proportion of patients with minimal/no pain at 12 and 24 months;
increased mean EQ-5D scores; and
increased proportion of patients achieving composite of treatment success in pain and function through 24 months.

Taken together, the above referenced data indicate improved treatment outcomes for early intervention with cell therapy in patients with CLBP, in particular intervention before 68 months of CLBP.

### Reduction in Axial Pain

Both treatment with MPC (n=15) and MPC+HA (n=14) reduced axial pain in patients with MRI-defined nerve root compression (Tables 1 and 2). These data support a method of reducing axial pain in subjects by administering MLPSCs, in particular in subjects with MRI-defined nerve root compression.

TABLE 1
Mean change in VAS pain score in patients
with nerve root compression at baseline.

| Treatment Group | N | 3 Months | 6 Months | 12 Months | 24 Months |
|---|---|---|---|---|---|
| Saline | 13 | −2.1 | −2.5 | −2.7 | 1.5 |
| 6M MPC + HA | 14 | −25.2 | −38.6 | −38.9 | −24.1 |
| 6M MPC | 15 | −27.1 | −27.1 | −25 | −21.7 |

TABLE 2
50% VAS pain score responders to treatment in patients
with nerve root compression at baseline.

| Treatment | | | | | | | | |
|---|---|---|---|---|---|---|---|---|
| Group | N | 3 Months | | 6 Months | 12 Months | | 24 Months | |
| Saline | 13 | 1 | (8%) | 1 (8%) | 2 | (15%) | 0 | (0%) |
| 6M MPC + | 14 | 9 | (64%) | 11 (79%) | 10 | (71%) | 7 | (50%) |
| HA | | | | | | | | |
| 6M MPC | 15 | 4 | (27%) | 10 (67%) | 7 | (47%) | 7 | (47%) |

What is claimed is:

1. A method of reducing pain or reducing opioid use or increasing EQ-5D score, the method comprising administering to the subject a composition comprising mesenchymal lineage precursor or stem cells (MLPSCs), wherein the subject is using an opioid.

2. The method of claim 1, wherein the composition comprises hyaluronic acid (HA).

3. The method of claim 1 or claim 2, wherein the subject is using an opioid for pain.

4. An opioid sparing composition comprising mesenchymal lineage precursor or stem cells (MLPSCs).

5. The composition of claim 4, further comprising hyaluronic acid (HA), preferably 1% HA.

6. The composition of claim 4 or claim 5, wherein the opioid sparing composition is administered to a subject using an opioid, preferably, wherein the subject is using the opioid for pain.

7. The method according to any one of claims 1 to 3 or the composition of claim 6, wherein of the pain is chronic pain.

8. The method according to any one of claims 1 to 3 or the composition according to any one of claims 4 to 7, wherein pain is lower back pain, preferably wherein the lower back pain is associated with a degenerated disc.

9. The method or composition of claim 7, wherein the lower back pain is associated with an intervertebral disc, preferably wherein the disc has a disc height that is not substantially reduced compared to that of an adjacent healthy disc in the subject.

10. The method or composition of claim 7, wherein the lower back pain is non-radicular in origin and/or is associated with one or more of:
an intervertebral disc herniation up to a 3 mm protrusion.
nerve ingrowth into an intervertebral disc.
inflammation in an intervertebral disc.

11. The method or composition claim 10, wherein the nerve ingrowth or inflammation is in the intervertebral disc space, or the nucleus pulposus, or the annulus fibrosis of the intervertebral disc.

12. The method according to any one of claims 1 to 3 or 6 to 11 or the composition according to any one of claims 5 to 11, wherein opioid use is reduced 1 month, preferably 3 months after administration of the composition.

13. The method according to any one of claims 1 to 3 or 6 to 11 or the composition according to any one of claims 5 to 11, wherein opioid use is reduced 6 months, preferably 12 months, more preferably 18 months, even more preferably 24 months after administration of the composition.

14. The method according to any one of claims 1 to 3 or 7 to 11 or the composition according to any one of claims 5 to 11, wherein opioid use is reduced for at least 12 months, preferably at least 24 months after administration of the composition.

15. The method according to any one of claims 1 to 3 or 6 to 14 or the composition according to any one of claims 5 to 14, wherein opioid use is reduced relative to the subjects average baseline morphine equivalent dose prior to administering the composition.

16. The method according to any one of claims 1 to 3 or 6 to 15, wherein the subject has been using an opioid for at least 1 month, preferably at least 3 months, more preferably at least 6 months prior to administering the composition.

17. The method according to any one of claims 1 to 3 or 6 to 16, wherein the subjects opioid use is reduced by about 20%, about 30%, about 40% relative to their baseline opioid use prior to administering the composition.

18. The method according to any one of claims 1 to 3 or 6 to 17, wherein the subjects mean VAS score is reduced relative to their VAS score prior to administering the composition.

19. The method according to any one of claims 1 to 3 or 6 to 18, wherein the subjects visual analog scale (VAS) pain response is 30%, preferably 50%.

20. The method according to any one of claims 1 to 3 or 6 to 19 or the composition according to any one of claims 5 to 19, wherein the opioid is morphine.

21. The method according to any one of claims 1 to 3 or 6 to 20, wherein the subjects mean EQ-5D score is increased from baseline.

22. The method according to any one of claims 2 to 3 or 6 to 20, wherein the subject achieves a ODI 10 point function response, preferably a 15 point ODI function response.

23. The method according to any one of claims 1 to 3 or 6 to 22, wherein the subject has no significant pain 12 months, preferably 24 months after administering the composition.

24. The method according to any one of claims 1 to 3 or 6 to 23, wherein the subject discontinues opioid use 12 months to 24 months after administering the composition.

25. The method according to any one of claims 1 to 3 or 6 to 24, wherein the subject has an ODI score between 25 and 70%, between 30 and 60%.

26. The method according to any one of claims 1 to 3 or 6 to 25 or the composition according to any one of claims 5 to 15, wherein the MLPSCs are STRO-1+.

27. The method according to any one of claims 1 to 3 or 6 to 26 or the composition according to any one of claims 5 to 15 or 26, wherein the MLPSCs are mesenchymal stem cells (MSCs).

28. The method according to any one of claims 1 to 3 or 6 to 27 or the composition according to any one of claims 5 to 15 or 26 or 27, wherein the cells are allogeneic and/or culture expanded, preferably, wherein the cells are TNAP+ before they are culture expanded.

29. The method according to any one of claims 1 to 3 or 6 to 28 or the composition according to any one of claims 5 to 15 or 25 to 28, wherein the cells have been cryopreserved.

30. The method according to any one of claims 1 to 3 or 6 to 29, which comprises administering between 1×10⁷ and 2×10⁸ cells.

31. The composition according to any one of claims 5 to 15 or 25 to 29, which comprises between 1×10⁷ and 2×10⁸ cells.

32. The method according to any one of claims 1 to 3 or 6 to 30 or the composition according to any one of claims 5 to 15 or 25 to 29 or 31, wherein the composition comprises human bone marrow-derived allogeneic mesenchymal precursor cells (MPCs) isolated from bone mononuclear cells with anti-STRO-3 antibodies, expanded ex vivo, and cryopreserved.

33. The method according to any one of claims 1 to 3 or 6 to 32 or the composition according to any one of claims 5 to 15 or 25 to 29 or 31 or 32, wherein the subject has been experiencing pain for between 6 and 68 months.

34. The method according to any one of claims 1 to 3 or 6 to 33 or the composition according to any one of claims 5 to 15 or 25 to 29 or 31 to 33, wherein the subject has not been experiencing pain for more than 68 months.

35. The method according to any one of claims 1 to 3 or 6 to 33 or the composition according to any one of claims 5 to 15 or 25 to 29 or 31 to 34, wherein the pain is axial pain.

36. The method or composition according to claim 35, wherein the axial pain is due to nerve root compression defined by MRI.

37. The method according to any one of claims 1 to 3 or 6 to 36, wherein the subject discontinues opioid use 36 months after administering the composition.

38. The composition according to any one of claims 5 to 15 or 25 to 29 or 31 to 34, wherein the composition discontinues opioid use in a subject 36 months after administration.

39. Use of a composition according to any one of claims 5 to 15 or 25 to 29 or 31 to 34 in the manufacture of a medicament for discontinuing opioid use by a subject, wherein the opioid use is discontinued 36 months after administration of the composition.

40. Use of claim 39, wherein the subject has chronic pain.

41. Use of claim 40, wherein the subject has chronic lower back pain.
